(19) United States
(12) Patent Application Publication
(10) Pub. No.: US 2006/0161443 A1
(43) Pub. Date: Jul. 20, 2006

(54) Systems and methods for collecting and managing animal-related information

(75) Inventor: R. Randall Rollins, Atlanta, GA (US)

Correspondence Address: JOHN S. PRATT, ESQ;KILPATRICK STOCKTON, LLP, 1100 PEACHTREE STREET, ATLANTA, GA, 30309, US

(73) Assignee: Lladnar Technology Co, LLC

(21) Appl. No.: 11/036,829

(22) Filed: Jan. 14, 2005

(51) Int. Cl. G06Q 99/00

(52) U.S. Cl. 705/1

(57) ABSTRACT

The invention relates to methods and systems for collecting and managing animal-related information. One aspect of an embodiment of the invention includes a method for collecting and managing animal-related data. The method includes logging in a network user, and receiving a selection of an animal management function. The method also includes defining an animal-related group and related data, and defining at least one template based at least in part on animal-related data. Furthermore, the method includes locating at least some of the desired animal-related data, and processing at least some of the desired animal-related data. Moreover, the method includes managing at least some of the desired animal-related data.

### FIELD OF THE INVENTION

This invention is generally directed to systems and methods for collecting and managing information. More particularly, the invention relates to methods and systems for collecting and managing animal-related information.

### BACKGROUND

In the animal processing industry, such as the cattle processing industry, an animal can be generally tracked and monitored from its conception through various stages of its growth through to processing and consumption of the animal. In the course of this monitoring, various participants in the industry, including ranchers, stockmen and stockyard operators, and meat packers, can collect and analyze various data pertaining to a particular animal. In some instances, the analysis of such data can lead to improved animal quality by regulating production costs and expenses. For example, industry participants can be interested in animal-related data such as “lifecycle” data. Analysis of such data can indicate what to do at each stage of an animal's life to achieve desired results, that is, obtaining maximum yield and a maximum price.

Each participant in the industry may collect data of particular interest for analysis of their own part in the processing cycle. For example, a rancher may be interested in ascertaining and monitoring which bulls produce the best offspring. This determination can be facilitated by tracking which bull's

offspring produce the best final product, which in this industry may be the final delivered food product as measured by the tenderness of the meat or by the grades of leanness resulting in price premiums. Alternatively, a stockman may be interested in determining which nutritional approaches produce the best and fastest weight gain in the animal. Moreover, he or she may be interested in knowing whether different approaches to achieving rapid weight gain are better suited to different breeds of livestock. Similarly, the meat packer, who receives animals and produces the outgoing food product, can make assessments of the quality of the incoming animals and can relate it to the quality of the final food product. A meat packer may also be interested in evaluating which breeds of animals handle climates better than others, or if different foods are better for particular breeds, etc.

Conventional attempts to collect and manage data for each animal and making the information available to other participants in the industry have led to some improvements the entire processing cycle. However, each industry participant may have only a limited understanding of the implications of the data that he or she has collected and how those implications may affect his or her own role in the processing cycle.

In at least one conventional system, the data collection lacks structure and consistent management. This can cause errors in data collection, and inconsistencies in subsequent results based on flawed data. Furthermore, the entire lifecycle of an animal cannot be traced to determine many factors, such as what feed was given to particular animal at a particular age.

Therefore, there is a need for improved systems and methods for collecting and managing animal-related information, such as an improved integrated animal data collection and management system.

There is a further need for providing systems and methods for collecting information related to an animal from its conception to production of a final food product, and providing a meaningful tool for determining which actions create a desired result.

There is a further need for providing systems and methods for tracking an animal from its conception to production of a final food product.

There is a further need for providing systems and methods for managing information related to an animal from its conception to production of a final food product.

There is a further need for providing systems and methods for sharing information related to an animal with various industry participants from the animal's conception to production of a final food product.

There is a further need for providing systems and methods for improved animal-related data collection and management.

There is a need for systems and methods providing a uniform data collection language adapted to foster more consistent food products, resulting in higher price premiums for industry participants.

### SUMMARY OF THE INVENTION

Accordingly, systems and processes according to various aspects and embodiments according to the invention address at least some or all of the above needs. They do at least in part by automating animal-related information collection, tracking, sharing, and management among a variety of users, including users at collection sites of interest as well as users who wish to access the data that has already been collected. These systems and processes can allow users to collect various data of interest, input such data into an automated system for processing, storing, and managing the data, and manage the data in accordance with a particular user's needs as dictated by which segment of the animal industry the user participates in. These systems and processes can facilitate data collection, organization, and distribution through integration of data collection devices, data input devices and operations, data communication mechanisms, database storage and management applications, and data access and processing applications. In addition, such systems and processes, on request by a user, can invoke and implement executable computer code that greatly facilitates the user's interaction with the system through use of convenient and intuitive graphical user interfaces (GUIs). Systems and processes according to various aspects and embodiments can operate with a various operating systems on a variety of platforms, including but not limited to Windows®-based systems, UNIX®-based systems, AIX®-based systems, processor-based platforms, personal computers, handheld computers, mainframe computers, parallel processing platforms, and supercomputers.

One aspect of systems and processes according to various embodiments of the invention focuses on methods for collecting animal-related information. One method according to an embodiment of the invention includes receiving previously stored animal-related data, and receiving a selection of at least a portion of the previously stored animal-related data. The method also includes generating a data structure adapted to be associated with new animal-related data, and receiving an identifier for an animal, wherein the identifier is associated with the data structure. Morover, the method also includes receiving new animal-related data for association with the data structure.

Another aspect of an embodiment of the invention includes methods for managing animal-related information that has been previously collected. One method according to an embodiment of the invention includes logging in a network user, and receiving a selection of an animal management function. The method also includes defining an animal-related group and related data, and defining at least one template based at least in part on animal-related data. Furthermore, the method includes locating at least some of the desired animal-related data, and processing at least some of the desired animal-related data. Moreover, the method includes managing at least some of the desired animal-related data.

Another aspect of an embodiment of the invention includes systems for collecting and managing animal-related information. One system according to an embodiment of the invention includes a processor adapted to: receive previously stored animal-related data, receive a selection of at least a portion of the previously stored animal-related data, generate a data structure adapted to be associated with new animal-related data; receive an identifier for an animal, wherein the identifier is associated with the data structure; and receive new animal-related data for association with the data structure.

Another system according to an embodiment of the invention includes a processor adapted to receive animal-related data associated with a plurality of animals. The system also includes a data storage device adapted to store at least some of the received animal-related data. The processor is further adapted to receive a request for a portion of the received animal-related data; and based on at least the request, provide at least some of the received animal-related data.

These example embodiments are mentioned not to limit or define the invention, but to provide examples of embodiments of the invention to aid the understanding thereof. Example embodiments are discussed in the Detailed Description, and further description of the invention is provided there.

### BRIEF DESCRIPTION OF THE DRAWINGS

These and other features, aspects, and advantages of the present invention are better understood when the following Detailed Description is read with reference to the accompanying drawings, wherein:

FIG. 1 illustrates an example system in accordance with an embodiment of the invention.

FIG. 2 illustrates an information collection component for the system shown in FIG. 1.

FIG. 3 illustrates an information collection component for the system shown in FIG. 1.

FIG. 4 illustrates an embodiment of an information collection process for the system shown in FIG. 1.

FIG. 5 illustrates another embodiment of an information collection process for the system shown in FIG. 1.

FIG. 6 illustrates another embodiment of an information collection process for the system shown in FIG. 1.

FIG. 7 illustrates an embodiment of an information management process for the system shown in FIG. 1.

FIG. 8 illustrates an embodiment of an information management process for the system shown in FIG. 1.

FIGS. 9-18 illustrate examples of graphical user interface screens for the method shown in FIG. 4.

FIG. 19 illustrates an example of a graphical user interface screen for the methods shown in FIGS. 5 and 6.

FIG. 20-35 illustrates examples of display screens for the methods shown in FIGS. 7 and 8.

### DETAILED DESCRIPTION OF DISCLOSED EMBODIMENTS

### System Architecture

The terms “data” and “information” are used interchangeably throughout the text of this specification and the meanings of both terms are intended to be similar in scope and interpretation.

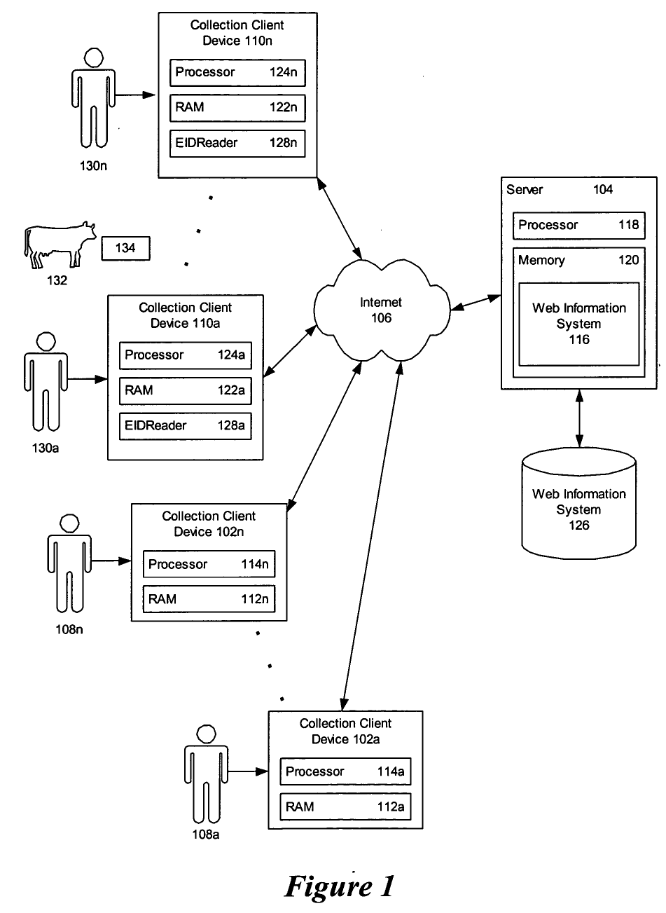

Referring now to the drawings in which like numerals indicate like elements throughout the several figures, FIG. 1 illustrates example system components for a system in accordance with an embodiment of this invention. The system 100 shown in FIG. 1 can comprise multiple access client devices 102a-n in communication with a server device 104 over a network 106, and operable by respective users 108a-n. The system 100 can also include multiple collection client devices 110a-n in communication with the server device 104 over the network 106. The network 106 shown can comprise the Internet. In other embodiments, network 106 can comprise other suitable types of networks, either wired or wireless, such as an intranet, local area network, wide area network, a telecommunications network, or broadcast network.

Each access client device 102a-n shown in FIG. 1 can comprise a computer-readable medium 112a-n. The embodiment shown in FIG. 1 can include a random access memory (RAM) 112a coupled to an access client processor 114a. The access client processor 114a can execute computer-executable program instructions stored in the memory 112a. Such processors can comprise a microprocessor, an Application Specific Integrated Circuit (ASIC), a state machine, or other suitable type of processor. Such processors can comprise, or may be in communication with, media such as computer-readable media that stores instructions and, when executed by the processor, can cause the processor to perform the steps described in some or all of the processes described herein.

Embodiments of computer-readable media can comprise an electronic, optical, magnetic, or other storage or transmission device capable of providing a processor, such as an access client processor 114a of access client device 102a, with computer-readable instructions. Other examples of suitable media can comprise a floppy disk, Compact Disk Read Only Memory (CD-ROM), magnetic disk, memory chip, Read Only Memory (ROM), Random Access Memory (RAM), an ASIC, a configured processor, all optical media, all magnetic tape or other magnetic media, or any other suitable medium from which a computer processor can read instructions or on which instructions, code, or other data can be stored. Also, various other forms of computer-readable media can transmit or carry instructions to a computer, including without limitation a router, private or public network, or other transmission device or channel, both wired and wireless. The instructions can comprise code from any suitable computer programming language including without limitation, for example, C, C⁺⁺, C#, Visual Basic, Java, Python, Perl, and JavaScript.

Access client devices 102a-n can also comprise a number of external or internal devices such as a mouse, a CD-ROM, a keyboard, a display, or other input or output devices. Examples of access client devices 102a-n are personal computers, media center computers, televisions, television set-top boxes, digital assistants, personal digital assistants, cellular phones, mobile phones, smart phones, pagers, digital tablets, laptop computers, Internet appliances, and other processor-based devices. In general, access client devices 102a-n can be any type of processor-based platform that can connect to a network 106 and that can interact with one or more application programs. Access client devices 102a-n can utilize any operating system, such as Microsoft® Windows® or Linux, capable of supporting one or more client application programs. For example, the access client device 102a shown can comprise a personal computer executing client application programs (also known as client applications). The client applications can be contained in memory 112a and can comprise, for example, a media player application, a presentation application, an Internet browser application, and any other application capable of being executed by an access client device 102a-n.

In one embodiment, an access client device 102a-n can comprise a laptop computer or portable processing device with an Internet browser application, such as Microsoft Explorer, Apple Safari, and Mozilla Firefox. The Internet browser application can provide network communications functionality for users operating the access client device 102a-n.

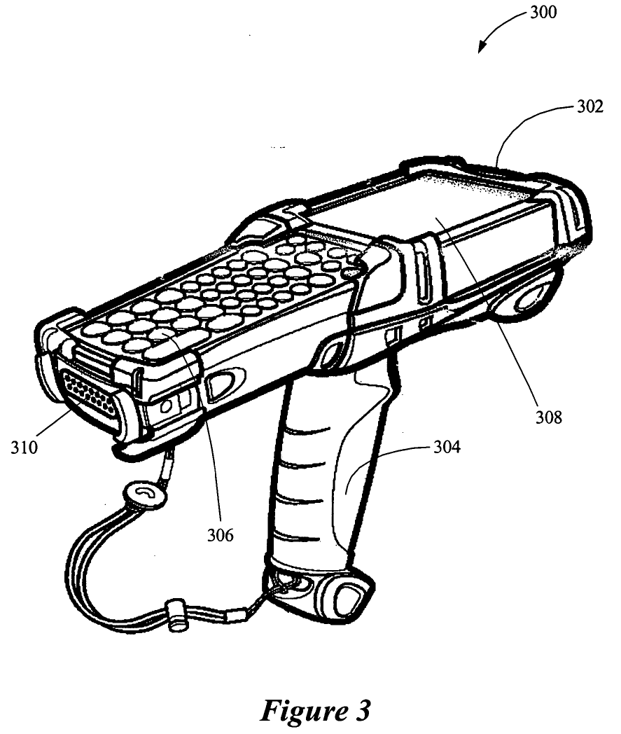

In one embodiment, a collection client device 110a-n can comprise a laptop computer or portable processing device with an Internet browser application, such as Microsoft Explorer, Apple Safari, and Mozilla Firefox. The Internet browser application can provide network communications functionality for users operating the collection client device 110a-n. The collection client device can also include or be functionally in communication with an associated data collection-type device such as a RFID reader, an integrated RFID reader/computer (described as 300 in FIG. 3 below), a bar code scanner, or a passive-type communication data device.

In at least one embodiment, a collection client device and access client device can be an integrated or single combined device capable of performing some or all of the functions of both a collection client device and an access client device.

Through the access client devices 102a-n can communicate over the network 106 with a server device 104, with other access client devices 102a-n, and with other systems and devices coupled to the network 106. As shown in FIG. 1, a server device 104 is also coupled to network 106. In the embodiment shown in FIG. 1, for example, an access site user 108a can use an access client device 102a to interact with the server device 104 and formulate a request for a specific set of livestock data, such as cattle-related data. The access client device 102a can send a request to the server device 104 via the network 106.

The server device 104 shown in FIG. 1 can comprise a server executing one or more application programs such as a web information system application program 116. Similar to access client devices 102a-n, the server device 104 shown in FIG. 1 can comprise a server processor 118 coupled to a computer-readable memory 120. Server device 104 is depicted in FIG. 1 as a single computer system, but can be implemented as a network of computer processors. Examples of a server device can include without limitation servers, mainframe computers, networked computers, processor-based devices, and similar suitable types of systems and devices. Access client processors 114a-n and server processor 118 can be any of a number of well known computer processors, such as processors from Intel Corporation of Santa Clara, Calif. and Motorola Corporation of Schaumburg, Ill.

Memory 120 on the server device 104 can store the web information system application program 116, such as the Web Cattle Information System (WCIS) application program to be distributed by LLADNAR Technology Company LLC of Atlanta, Ga. The web information system application program can include various functional modules (not shown) adapted to perform particular management tasks including, but not limited to, cattle management, semen management, medicine management, pasture management, payment management, and feedlot data uploading.

Each collection client device 110a-n shown in FIG. 1 can comprise a computer-readable medium. The computer-readable medium can comprise a random access memory (RAM) 122a-n coupled to a collection client processor 124a-n. The collection client processor 124a-n can execute computer-executable program instructions stored in the memory 122a-n. Such processors can comprise a microprocessor, an Application Specific Integrated Circuit (ASIC), a state machine, or other suitable processor. Such processors can comprise, or can be in communication with, media such as computer-readable media that can store instructions and, when executed by the processor, can cause the processor to perform some or all of the processed described herein.

Embodiments of computer-readable media can comprise an electronic, optical, magnetic, or other storage or transmission device capable of providing a processor, such as a collection client processor 124a-n of collection client device 110a-n, with computer-readable instructions. Other examples of suitable media can comprise a floppy disk, Compact Disk Read Only Memory (CD-ROM), magnetic disk, memory chip, Read Only Memory (ROM), Random Access Memory (RAM), an ASIC, a configured processor, all optical media, all magnetic tape or other magnetic media, or any other suitable medium from which a computer processor can read instructions or on which instructions, code, or other data can be stored. Also, various other forms of computer-readable media can transmit or carry instructions to a computer, including without limitation a router, private or public network, or other transmission device or channel, both wired and wireless. The instructions can comprise code from any suitable computer programming language including without limitation, for example, C, C⁺⁺, C#, Visual Basic, Java, Python, Perl, and JavaScript.

In the embodiment shown in FIG. 1, the server 104 can communicate with a web information system database 126. The web information system database 126 can receive information from the server 104, and store the information for subsequent access and retrieval by the server 104.

Collection client devices 110a-n can also comprise a number of external or internal devices such as a mouse, a CD-ROM, a keyboard, a display, or other input or output devices. Examples of collection client devices 110a-n are personal computers, media center computers, televisions, television set-top boxes, digital assistants, personal digital assistants, cellular phones, mobile phones, smart phones, pagers, digital tablets, laptop computers, Internet appliances, and other processor-based devices. In general, collection client devices 110a-n can be any suitable type of processor-based platform that can connect to a network 106 and that can interact with one or more application programs, such as the web information system application program 116. Collection client devices 110a-n can utilize any suitable operating system, such as Microsoft® Windows® or Linux, capable of supporting one or more client application programs. For example, the collection client device 110a shown can comprise a personal computer executing client application programs (also known as client applications). The client applications can be contained in memory 120a-n and can comprise, for example, a media player application, a presentation application, an Internet browser application, and any other application capable of being executed by a client device.

In the embodiment shown in FIG. 1, a collection client device 110a-n can include an EID reader application program 128a-n. The EID reader application program 128a-n can provide various data collection and management functionality for collection client users 130a-n.

Although a collection client user 130a, for example, can obtain data of interest about the livestock and enter such data into the collection client device 110a-n manually, in one embodiment of the present invention the collection client user 130a can obtain and enter data automatically. For example, an animal such as a bull 132 can be associated with an identifier 134. Data from the identifier 134 can be obtained by and entered into the collection client device 110a-n.

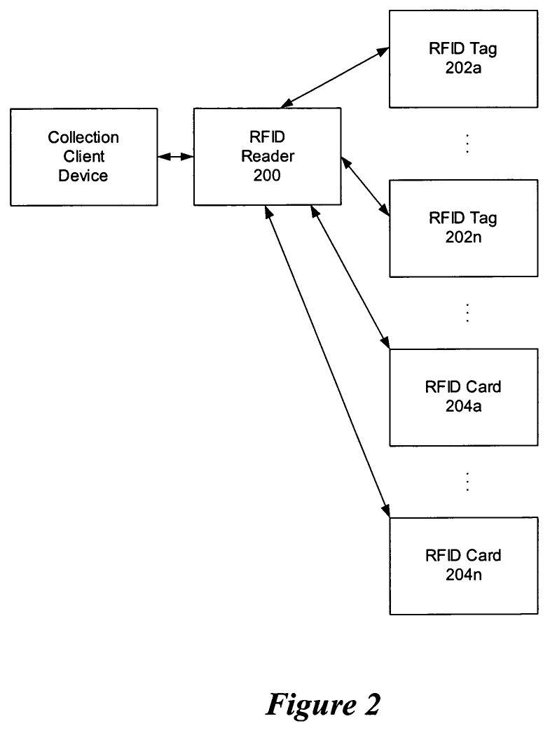

In one embodiment, a collection client device 110a-n can be associated with one or more suitable data collection-type devices such as a radio frequency identification (RFID) reader. In FIG. 2, a radio frequency identification (RFID) reader 200 can read or otherwise collect information from one or more RFID-type tags 202a-n and/or RFID-type cards 204a-n to obtain and enter data of interest for a collection client device 110a-n. As shown, a RFID reader 200 can be in communication, for example, either by a wire or wirelessly, with a respective collection client device 110a-n. Other suitable data collection-type devices can be utilized in accordance with embodiments of the invention including, but not limited to, bar code scanners, radio frequency emitting devices.

The RFID-type tags 202a-n shown in FIG. 2 can be conventional type radio frequency identification (RFID)-type tag devices. RFID-type tags 202a-n can be adapted to actively or passively provide information via a radio frequency to a corresponding RFID receiver or reader-type device.

The RFID-type cards 204a-n shown in FIG. 2 can be conventional type radio frequency identification (RFID)-type card devices. RFID-type cards 204a-n can be adapted to actively or passively provide information via a radio frequency to a corresponding RFID receiver or reader-type device.

In one embodiment, a collection client device 110a-n can be integrated with one or more suitable data collection-type devices. In FIG. 3, an integrated RFID reader/computer 300 can read or otherwise collect information from one or more RFID-type tags (similar to those shown in FIG. 2) and/or RFID-type cards (also similar to those shown in FIG. 2) to obtain and enter data of interest. The integrated RFID reader/computer 300 can be used in conjunction with or in lieu of the combination of a separate RFID reader, such as 200, and a separate collection client device 110a-n. The integrated RFID reader/computer 300 shown in FIG. 3 can comprise an RFID scanner 302, a handle 304 permitting a user to manually hold the device, an associated input device such as a keypad 306 for data entry, an interface such as a touch screen 308 for both viewing and entering or modifying displayed data, a communications port 310 for sending and receiving data to and from a desktop personal computer (such as a collection client device 108a-n) or across a network 106 to a server device 104, a processor (not shown), and a memory (not shown). For example, a user operating the integrated RFID reader/computer 300 can direct or otherwise move the RFID scanner 302 towards or in the vicinity of a RFID-type tag or card associated with an animal such as a cow or bull. Data from the RFID-tag or card can be collected, transmitted to an associated processor, and stored in an associated memory. The user can view some or all of the collected and/or stored data via the touch screen 308, and input commands or other data via the keypad 306. When needed, the communications port 310 can transmit some or all of the collected and/or stored data, or other data stored in the processor or memory to a remote location via the network 106 to the server device 104. For example, data can be transmitted to the server device 104 via a local wireless network, such as a WI-FI-type network. By way of another example, data can be transmitted to the server device 104 via a cellular data network, such as a GPRS-type network. In yet another example, data can be transmitted to the server device 104 via a conventional wired network, such as a cable or PSTN-type network.

Although the processes described herein are described in relation to clients and a server or servers, a client can perform any or all of the processes described as being performed by the server or servers. Similarly, a server or servers can perform any or all of the processes described as being done by a client. Moreover, although the processes described herein are also described in relation to access clients and collection clients, these separate functional processes need not be embodied in separate hardware arrangements, but can coexist or otherwise be integrated on the same set of hardware.

Other embodiments of the present invention can comprise systems having different architecture than that which is shown in FIGS. 1-3.

Processes

A. Data Collection

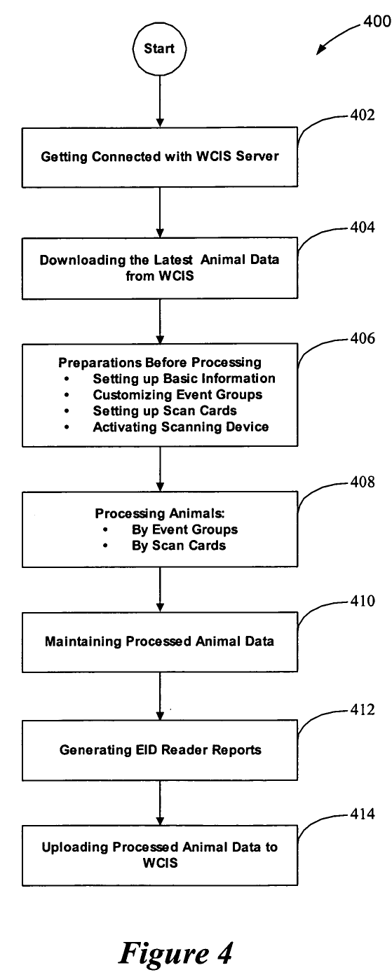

FIG. 4 illustrates a method according to an embodiment of the invention. In the embodiment shown, the method 400 collects and manages animal-related information. The method 400 shown can be implemented by an EID reader application program, such as 128a-n shown in FIG. 1. In one embodiment, a collection device user 130a-n can operate a client collection device 110a-n, and implement the method 400 described below to collect information related to animals, such as a herd of cattle wearing a respective RFID device.

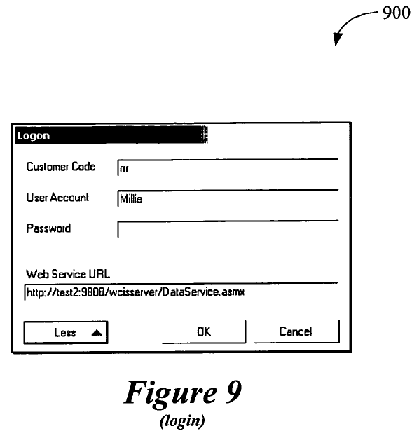

The method 400 begins at block 402, in which a collection client device connects with a server. In the embodiment shown, a collection client device 110a-n connects with a server 104 by facilitating communication with the server 104 shown in FIG. 1. Communication with the server 104 can include execution of one or more authentication and user identification verification methods and routines prior to permitting access by the collection client device 110a-n to functionality available from the server 104. When the collection client device 110a-n is authenticated, or an identity of a collection client device user 110a-n is otherwise verified, then a collection client device user 130a-n can access some or all of the functionality available from the server 104 via the collection client device 110a-n. As shown and described in FIGS. 9-18 below, various graphical user interfaces (GUIs) corresponding with respective functionality associated with the EID reader application program 128a-n can provide a user 130a-n with access to features associated with the EID reader application program 128a-n. Functionality associated with the EID reader application program 128a-n can include, but is not limited to, data management, latest animal management, basic information management, processing event management, processing event group management, scan card, EID reader serial port settings, individual processing animal card, result data processing, generate EID reader reports, download/upload data to/from a web information system application program, and exit program. Other functionality according to other embodiments of the invention can be provided.

Block 402 is followed by block 404, in which recent animal-related data is downloaded from the server. In the embodiment shown, the collection client device 110a-n can download some or all recent animal-related data such as cattle-related information from the server 104. Activities associated with downloading can include, but are not limited to, registering a particular download job on the server, synchronizing information, locating information on an associated database such as a web information system database 120, sorting the information onto an associated web site from a database, downloading a file containing information from an associated web site to a collection client device 110a-n, unzipping information from a downloaded file, and inserting downloaded information into memory or another suitable data storage device. Recent cattle-related information can be information previously stored, collected, or otherwise downloaded to the server. Cattle-related information can include, but is not limited to, animal classification, herd name, ranch code, medicine, bull/sire group, and other basic information associated with one or more cattle.

Prior to downloading recent information to a collection client device 110a-n, an additional set of authentication and/or user identification verification methods and routines can be implemented. Once the collection client device is authenticated, or when the identity of a collection client device user 130a-n is verified, then cattle-related information can be downloaded from the server 104, or via the server 104 from the web information system database 126 or other associated data storage device.

In one embodiment, relatively efficient downloading of information can be performed. When cattle-related information has previously been downloaded from the server 104 to a particular collection client device 110a-n, the server 104 can transmit only a portion of the cattle-related information that has changed or that has been updated since the previous download. In this manner, data transfer time and system resources can be conserved.

In one embodiment, cattle-related information based on a particular ranch or other group can be downloaded. For example, a graphical user interface (GUI) can display a list of ranch codes via a display device associated with the collection client device 110a-n. Using an associated input device, a user 130a-n can indicate one or more ranch codes to obtain cattle-related information for, and the server 104 can transmit corresponding cattle-related information to the collection client device 110a-n for the selected ranch codes.

Block 404 is followed by block 406, in which the collection client device is adapted to receive new animal-related data. In the embodiment shown, the collection client processor 124a-n receives the downloaded cattle-related information from the server 104, and sets up basic information for collecting additional cattle-related information via the respective collection client device 110a-n. The basic information can be set up depending on a particular process or processes to be performed. By way of example, processes can include adding a new medicine, processing animals for a marking and branding session, and setting up scan cards for a particular data collection period. Basic information can include, but is not limited to, animal classification, ranch code, herd name, medicine, and bull/sire group. The processor 124a-n can organize some or all of the downloaded cattle-related information using some or all of the types of basic information.

For example, the collection client processor 124a-n can utilize a series of graphical user interfaces such as predefined windows to organize, collect, and manage information received from the server 104. A user 130a-n can then navigate through the various windows to view and modify information as needed. In the following examples, particular user interfaces such as windows in accordance with embodiments of the invention are defined and described for related information.

In one example, the collection client processor 124a-n can setup a user interface such as a window to list all available animal classifications downloaded from the server 104. The window can be accessed via functionality associated with the EID reader application program, such as a graphic user interface with icons for basic information management and animal classifications. When selected, the window can be displayed by the processor 124a-n for viewing and selection of particular data by a respective user 130a-n. The window can include animal classifications including, but not limited to, 1^{st} calf heifer, bred heifer, breeding bull, bull calf, exposed heifer, heifer, heifer calf, mature cow, RPL heifer, steer, steer calf, and yearling bull. Other information associated with each animal classification can be displayed, such as additional description of each animal, i.e. whether the animal is a male or female. Likewise, data reporting based on some or all of the above classifications and information can be performed when needed.

In another example, the collection client processor 124a-n can setup a window to list all available ranch codes downloaded from the server 104. The window can be accessed via functionality associated with the EID reader application program, such as a graphic user interface with icons for basic information management and ranch codes. When selected, the window can be displayed by the processor 124a-n for viewing and selection of particular data by a respective user 124a-n. The window can include user-defined or predefined ranch codes and/or names, for example, RRBC and RRCA. Other information associated with each ranch code can be displayed, such as additional description of each ranch, a ranch number, ranch name, and a creation date.

In yet another example, the collection client processor 124a-n can setup a window to list all available herd names downloaded from the server 104. The window can be accessed via functionality associated with the EID reader application program, such as a graphic user interface with icons for basic information management and herd names. When selected, the window can be displayed by the processor 124a-n for viewing and selection of particular data by a respective user 130a-n. The window can include user-defined or predefined herd names and/or herd labels such as 108BBR01. Other information associated with each herd name can be displayed, such as additional description of each herd, a ranch code, ranch name, and a creation date.

In one example, a new medicine can be added by a collection client user. The collection client user 130a-n can access a graphical user interface that can display previously stored medicinal information. The collection client user 130a-n can enter information associated with a new medicine into an associated data entry window. The information can include, but is not limited to, a name of the medicine, a treatment for which the medicine is directed, a period of time during which the medicine is effective, dosage information, and other medicinal-related information. Once the information is entered, the collection client processor 124a-n can store the information in memory or another suitable data storage device for subsequent retrieval.

In another example, the collection client processor 124a-n can setup a window to list all available medicines downloaded from the server 104. The window can be accessed via functionality associated with the EID reader application program, such as a graphic user interface with icons for basic information management and medicine. When selected, the window can be displayed by the processor 124a-n for viewing and selection of particular data by a respective user 130a-n. The window can include user-defined or predefined medicines including, but not limited to, Covexin 8, Curatrem, Elite 4HS, Fortress8, Ivomec, Patriot Fly Tags, Triangle 4HS, Trichguard V5L, and Vibrio Lepto. Other information associated with each medicine can be displayed, such as additional description of each medicine, usage, and utility.

In yet another example, the collection client processor 124a-n can setup a window to list all available bull/sire groups downloaded from the server 104. The window can be accessed via functionality associated with the EID reader application program, such as a graphic user interface with icons for basic information management and bull/sire groups. When selected, the window can be displayed by the processor 124a-n for viewing and selection of particular data by a respective user 130a-n. The window can include user-defined or predefined bull/sire groups such as BC-BM0306-2. Other information associated with each bull/sire group can be displayed, such as additional description of each bull/sire group, ranch code, and creation date.

Depending on the type, quantity, and/or quality of the information, other suitable graphical user interfaces and windows can be utilized in accordance with embodiments of the invention to organize, collect, and manage information from the server. In at least one embodiment, a data structure such as a template can be utilized to organize a particular type of information, such as events or sets of related events. Other data structures can be used in accordance with embodiments of the invention.

For example, a collection client device 110a-n can be adapted to receive new animal-related data associated with a particular event or event group, such as setting up a template for collecting information associated with marking and branding a particular group of animals. An event can be any activity related to animal data management, such as cattle-related data management. An event can include, but is not limited to, artificial insemination (AI), birth, body condition, breeding, brucellosis, death, implant, pregnancy, pregnancy check, purchase, sale, transfer, vaccination, and weaning. An event group is a set of related events, such as Commercial Bull Calves, Commercial Heifer Calves, Commercial Steer Calves, Mature Cows, and Registered Herd Bull Calves.

The collection client processor 124a-n can initially setup and display a window to list all available events and event groups based in part on at least data downloaded from the server 104. The window can be accessed via functionality associated with the EID reader application program, such as a graphic user interface with icons for basic information management and event groups. When selected, the window can be displayed by the processor 124a-n for viewing and selection of particular data by a respective user 130a-n.

For example, a collection client user 130a-n can select the event group of interest from the groups displayed in the user interface window. This action can open a new user interface window that permits the collection client user 130a-n to select events from a list of available events for association with that particular group. For example, available events under an event group associated with the Marking and Branding process can include, without limitation, AI Breed, AI Bull Cane, AI Bull Name, AI Bull Visual, AI Date, AI Tech, Animal Classification, Birth Date, Body Condition Score, Body Condition Score—Bull BR, Body Condition Score—Calf Boost, Body Condition Score—Calf MB, Body Condition Score—Calf Wean, Body Condition Score—Cow Implant, Body Condition Score—Cow MB, WT—Calf MB, Hip Ht—Calf MB, Breeding Season, Breeding Year, MB Date, Sire Group, Year Code, Breed, Location, and Herd.

A user interface window can also display the available events that have already been selected for association with a particular event group along with default values assigned to those selected events. For example, Animal Classification can be set by default to Heifer Calf. To change a default setting for a particular selected event, the collection client user 130a-n may instruct the respective collection client processor 124a-n to edit the value of the selected event. This action can open a new user interface window that can display the available values for the highlighted selected event. The collection client user 130a-n can then choose the selected value from a set of available values and assign it to the chosen selected event. The collection client user 130a-n can repeat this action for each selected event displayed and thereby modify the current value of that selected event.

The collection client user 130a-n can also add events to the group in addition to the events that had been previously selected. From the list of available events, the collection client user 130a-n can designate an event to be added to a list of selected events. The collection client user 130a-n can then review and modify the default values of the newly added events in accordance with the discussion above.

Some selected events of interest, such as, for example, those pertaining to the height of an animal or to the breed, might possibly not be amenable to pre-assignment of default values, but rather can be better determined individually for each animal at the collection site. Thus, the collection client user 130a-n can determine the value of any selected event during processing. To determine a value in this way, the collection client user 130a-n can assign the selected event a value that can be recognized to indicate that the value will be assigned at the collection site during processing. For example, by default the assigned value of the selected event “Hip Ht-Calf MB” can be set to indicate that this value is assigned during processing. Thus, during the data collection process, the collection client user 130a-n obtains the value of this selected event and inputs it into the graphical user interface.

By way of further example, in preparing for a marking and branding session, a collection client user 130a-n can establish a different medicine regimen for different animal classifications. Thus, the collection client user 130a-n may want to vaccinate calves differently from cows and to give calves different parasite medicine than cows. The collection client user 130a-n, in this example, can associate the selected event “Vaccinate” with the different groups of animals, and then can assign different values to this selected event for the different groups to reflect the different medicine regimens. In establishing the value for this selected event, the collection client user 130a-n can choose the desired medicine for use in vaccinating the particular group of animals from a list presented by an appropriate user interface window. If the desired medicine did not appear in the list, then the collection client user could add the medicine to the system in accordance with the earlier discussion.

Once the collection client user 130a-n has determined which groups are of interest, associated the selected events of interest with each group, and assigned the proper value to each selected event, the collection client user 130a-n can then save the data in memory or another suitable data storage device for the customized event group.

In one embodiment, the collection client device can be adapted to receive new animal-related data including events associated with one or more “event groups.” For example, a “SALE” event group can be defined from events associated with sale of an animal, such as animal classification, WT-sale, sold to, breed, and year code. In some instances, some event groups can be applied to more than one type of animal, such as a particular type of cattle, i.e. heifers and bulls. In other instances, event groups can be combined together to form a compound event group, such as “WEANING+PREGNANCY CHECK.” Event groups can be predefined, defined or otherwise modified by users 108a-n, 130a-n, or defined or otherwise modified by the system 100 as needed. Event groups can be arranged together to form one or more cattle-related data collection and management processes. For example, SALE and TRANSFER event groups can be predefined to collect particular cattle-related data associated with the sale and subsequent transfer of a herd of cattle.

In one embodiment, a collection client device user 130a-n can add, delete, or otherwise modify an event group as desired. For example, a graphical user interface (GUI) can display a list of event groups via a display device associated with the collection client device 110a-n. Using an associated input device, a user 130a-n can select a particular event group to modify. The user 130a-n can then access data associated with the event group, and add, delete, or otherwise modify the data as needed. Data associated with each event group can include, but is not limited to, additional description of each event group, an event group value, and an event value. Modifications to the event group can be stored by the respective collection client processor 124a-n for subsequent retrieval or further modification.

In one embodiment, adapting the collection client device 110a-n to receive new animal-related data can include associating a unique electronic identification (EID) number for each event and/or event group. The EID number is a unique string of characters that can include letters, numbers, symbols, or any combination thereof. The EID number can be used to track the occurrence of a particular event or event group for a particular animal. For example, an electronic scan card can be setup to receive the input of an EID number. The electronic scan card can include the EID number, an indication of the type of event or event group, event detail or description, and a cross-reference to other EID numbers for related events of an event group.

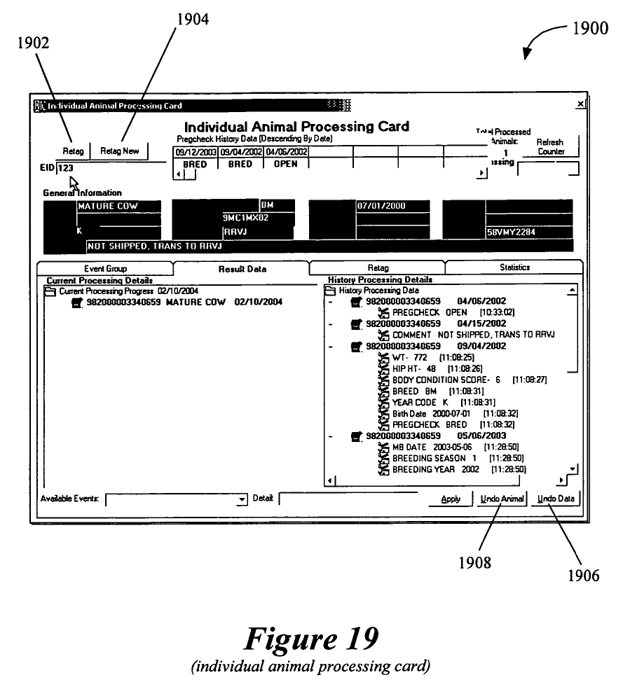

By way of yet another example, in addition to setting up groups of interest, a collection client user 130a-n can also set up an individual animal processing card or scan card, shown as 1900 in FIG. 19, prior to processing animals. For example, to set up an individual animal processing card or scan card 1900 for a marking and branding group, the system 100 can present a user interface window. This user interface window permits the collection client user 130a-n to input information about the electronic identification (EID) number for the scan card and the single event or the event group linked to each scan card 1900. In particular, the collection client user 130a-n can enter the EID number for a particular card of interest. The entered EID number can be used only with that particular scan card. The system 100 can then permit the user to associate the card with either a single selected event or an event group. This action can cause a user interface window to present a list to the collection client user 130a-n to choose the particular selected event or the particular event group as appropriate. For example, if a scan card 1900 is to be associated with the event group M & B—Commercial Heifer Calves, the collection client user 130a-n can highlight a radio button next to the Event Group entry in the user interface window. This action can cause the user interface window to present a list of event groups from which the collection client user 130a-n can choose the M & B—Commercial Heifer Calves event group. Once the collection client user 130a-n has associated the EID number with the appropriate event group or selected event, the collection client user 130a-n may inform the system 100 that the association is complete, which permits the collection client user 130a-n to input information for another scan card. After the collection client user 130a-n has entered information pertaining to all the desired scan cards 1900, the collection client user 130a-n may close the user interface window and return to a main user interface window.

After preparing the system 100 to receive data pursuant to a particular measurement regimen, the collection client user 130a-n can activate scanning devices to begin collecting data of interest. The scanning devices can include a stick reader and a weight scale and can be activated by associating a device with a communication port on the collection site computer. To activate the scanning devices, the system 100 can present a user interface window that permits the collection client user 130a-n to associate a particular scanning device with a particular communications port through which the scanning device may communicate with the respective collection client device 110a-n. The user 130a-n can associate a particular scanning device with a particular communication port by choosing the particular scanning device from a list within the user interface window. After having selected the particular scanning device of interest, the user 130a-n can then choose a communications port from a list and can instruct the system 100 to associate the selected scanning device with the selected communications port.

After associating the scanning devices with the desired communication ports, the collection client user 130a-n can provide the system 100 with general processing information prior to collecting data. Such general information can include without limitation the first day of processing, the process name, the ranch where the livestock to be processed may be found, the herd or herds where the livestock to be processed may be found, the herd or herds where the livestock will stay after processing, and one or more types of animals to be processed.

In one embodiment, adapting the collection client device 110a-n to receive new animal-related data can include activating the collection client device 110a-n and an associated weight detection device. Suitable collection client devices can include, but are not limited to, an Aginfolink Stick Reader, and an AllFlex Stick Reader. Suitable weight detection devices can include, but are not limited to, a weight scale device, a Tru-Test 701, 702, 703 scale, and a Tru-Test AG500/03 scale. The respective collection client processor 124a-n can setup a window to list all available collection client devices and an associated weight detection devices. The window can be accessed via functionality associated with the EID reader application program, such as a graphic user interface with icons for selecting devices. When a particular device is selected by a user 130a-n, the window can display the selection and any other information related to the selected device.

Block 406 is followed by block 408, in which one or more animals are processed. After a client collection device 110a-n adapted to receive new animal-related information, such as described above in block 406, the client collection device 110a-n can be utilized to process one or more animals, such as a herd of cattle. Depending on the circumstances or a user's decision, animals can be processed by scan card, event group, single events, or any combination thereof.

Initially, general information associated with a particular group of animals can be input via the client collection device 110a-n, such as general information about a particular herd of cattle. General information can include, but is not limited to, a process name, a process time, working ranch, working herd, destination ranch, destination herd, animal classification. The collection client processor 124a-n can setup a window to prompt a respective client collection device user 130a-n to input general information through one or more graphical user interfaces (GUIs) such as webpages. The window can be accessed via functionality associated with the EID reader application program, such as a graphic user interface with an icon for processing. Using an associated input device, a user 130a-n can input, or otherwise modify general information into the webpages. The input information can be stored by the respective processor 124a-n for subsequent retrieval.

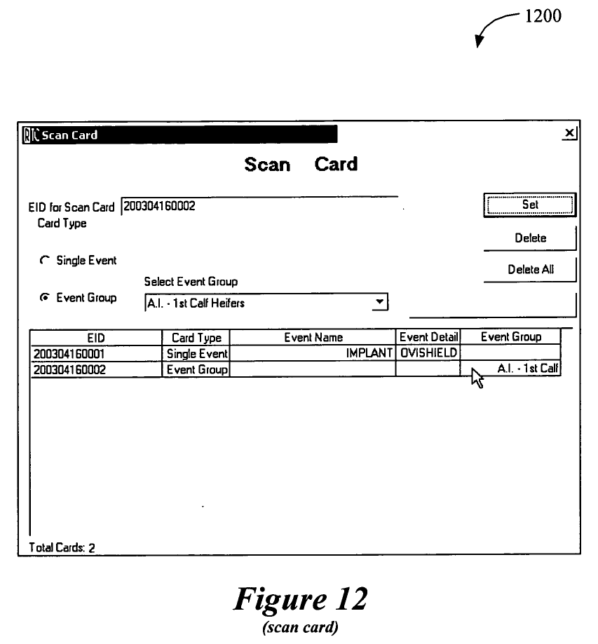

After general information is entered, the client collection device user 130a-n can then begin to process animals using a selected data structure, such as a scan card or an event group. For example, a client collection processor 124a-n can initially setup a scan card based in part on at least a portion of the previously entered general information. An example of a scan card is shown in FIG. 12. The scan card shown in FIG. 12 includes various graphical user interfaces such as data input windows for receiving new animal-related information associated with the animals to be processed. The client collection device user 130a-n can then input a unique EID number associated with a particular animal in a corresponding graphical user interface window of a scan card. The user 130a-n can utilize a respective collection client device 110a-n and/or other suitable collection data input device such as an integrated RFID reader/computer to input an EID number. In any event, as each animal of a particular herd is processed, a different EID number for each animal of the herd can be input into a different scan card. EID numbers can be assigned to animals of a herd sequentially, at random, or by an algorithm or process. The scan cards can be stored by the respective client collection processor 124a in memory 122a-n or other suitable data storage device such as the web information system database 126.

In processing animals of different types according to the same process, the client collection processor 124a-n can present a predefined data entry window for entering information, and can accept the information entered into each scan card. For example, a user can select a particular scan card to process “Heifer Calves.” The client collection processor 124a-n displays a corresponding scan card for the respective collection client user 130a-n to input various information such as weight, hip height, and other measurements of interest. If the next animal to be processed is of a different type, the collection client user 130a-n can select another corresponding scan card, and can process the animal in accordance with information prompted by the scan card. After all the data has been collected for a group of particular animals, the client collection processor 124a-n can present summary pages of information including processing history for a particular animal.

In another embodiment, the collection client processor 124a-n can display a graphical user interface (GUI) window on an associated display device listing available event groups of animals within a particular herd of interest. A collection client user 130a-n can utilize a RFID reader/computer to scan an RFID tag 202a to identify a particular animal. The collection client processor 124a-n can then present a data entry window for the collection client user 130a-n to enter a value according to the process of interest. The data entry window can display some or all of the information received from the RFID tag 202a-n, and can provide the user 130a-n an option to modify the previously received information. For example, if the collection client user 130a-n is collecting weight information for a particular animal, the collection client processor 124a-n, after receiving information from a corresponding RFID tag 202a-n, can present a data entry window for entering a corresponding weight for the animal. Similar types of data entries can be made for other data of interest according to predefined information needs. After information has been collected for a particular animal, the collection client processor 124a-n can present summary pages of information including processing history for a particular animal.

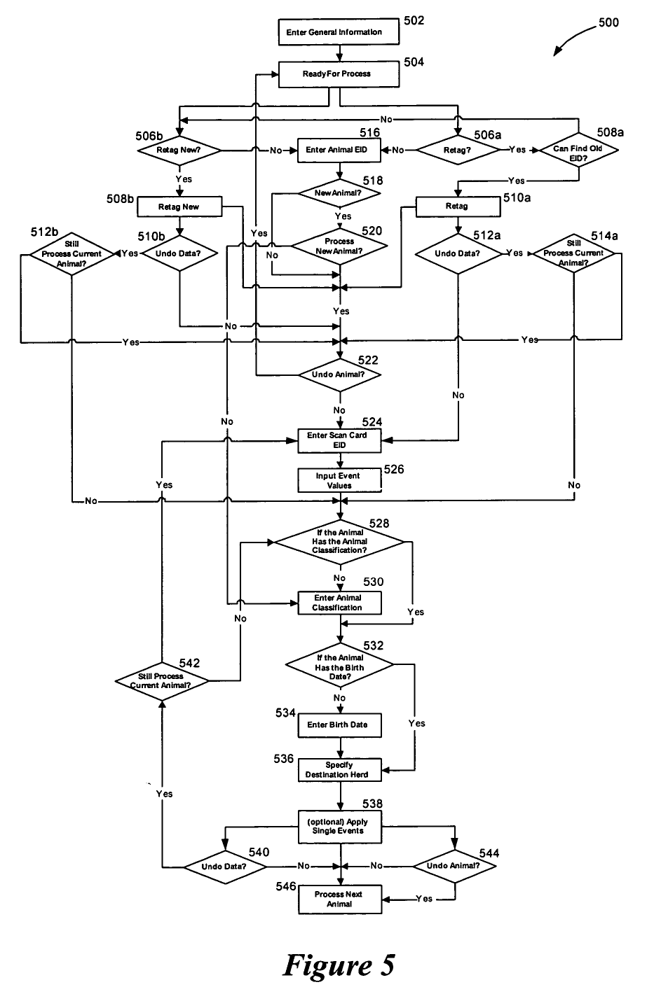

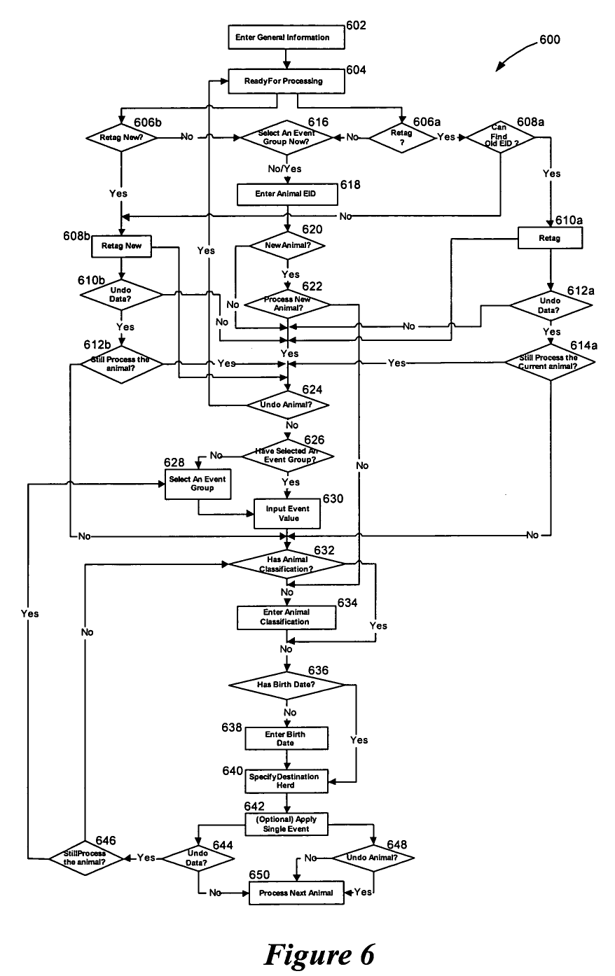

Other examples of methods for collecting and managing animal-related information using data structures such as scan cards and event groups are described in FIGS. 5 and 6 respectively.

After the user has completed processing all of the desired animals for a particular time, the collection client processor 124a-n can determine and display various calculation and statistical information based in part on at least the animal data collected. For example, some of the calculations by a collection client processor 124a-n can include, but are not limited to, a number of animals that were processed by a particular user such as a technician, a number of animals in a particular classification that were processed, a number of animals in a particular breed that were processed, a number of animals that failed or passed a particular medical test, a number of animals in a selected bull group, a number of animals that were culled for a specific reason, a number of animals put into a particular herd after processing, a number of animals that were purchased or given birth, a number of animals that were bred or open at the pregnancy check, a number of bulls that passed or failed a particular semen test, a number of calves that have a particular sire group, and a number of animals that were wet or dry. Statistical information can include, but is not limited to, statistics about the number of animal weights that fit a selected data range, breeding rates, and birth rates. For some or all of the calculations and statistical data, a collection client user 130a-n can select the calculations and statistics of interest, and the collection client processor 124a-n can process and display the selected calculations and/or statistics.

In addition to processing activities, the collection client processor 124a-n can also handle other circumstances, also called “special cases,” such as retagging an animal for which a RFID tag has been corrupted, canceling data that has been applied to a particular animal, and skipping an unexpected animal.

In one embodiment, when a RFID tag associated with a particular animal is corrupted, becomes unreadable to a scanning device, or is missing, a collection client user 130a-n can retag an animal with a new RFID tag. Initially, a collection client user 130a-n can obtain a replacement RFID tag, scan it with a scanning device such as a RFID reader/computer, and connect the RFID tag to the animal. The user can select a “Retag” option from a graphical user interface, and the collection client processor 124a-n can prompt the user to associate the new RFID tag with the old RFID tag and the data that had already been collected and associated with the old RFID tag. In some instances, if retagging an animal occurs during the processing of the animal, the collection client processor 124a-n can permit a collection client user 130a-n to complete processing for the particular animal.

During processing, information that has been applied to a particular animal may need to be deleted or otherwise modified. For example, a collection client user 130a-n can inadvertently scan information while in processing a particular animal. In such instances, the collection client processor 124a-n can provide the user 130a-n a graphical user interface option such as “Undo Animal” or “Undo Data” to correctly enter previously entered information.

In another embodiment, an animal that may not be intended to be processed with a desired group can nonetheless be part of a group of animals that has been processed, i.e. an “unexpected animal.” In this instance, when an unexpected animal is scanned, a flag or indication can be generated by the collection client processor 124a-n. The collection client user 130a-n can indicate to the collection client processor 124a-n that the particular animal is not to be processed with the desired group of other animals. The collection client processor 124a-n can then skip processing information associated with the unexpected animal, and maintain the information separate from the information associated with the desired group of other animals.

Block 408 is followed by block 410, in which the collected animal-related information is maintained. After all of the animals for a particular group have been processed and corresponding animal data has been collected, the collection client processor 124a-n can permit a collection client user 130a-n to maintain, review, or otherwise modify the collected information. The collected data can be maintained, reviewed, or modified by the collection client processor 124a-n, and stored in memory 122a-n or other suitable storage device such as the web information system database 126 for subsequent transfer or retrieval.

In one embodiment, a collection client processor 124a-n can provide a graphical user interface for the user to sort the information by various criteria. A collection client user can sort the collected information based on one or more criteria. For example, the collection client user can sort the data according to the date the information was collected and/or processed. Alternatively, a collection client user can sort data based on at least an EID number and/or one or more events or event groups. The collection client processor 124a-n can present a user with various search filters to perform the desired sorting. The collection client user can then assign values to various search filters as needed. The collection client processor 124a-n can then apply some or all of the defined filters to determine matching or sorted information.

In one embodiment, a collection client processor 124a-n can provide a graphical user interface for the user to change details of previously collected information, add information to the previously collected information, delete some or all of the previously collected information, and determine and eliminate some or all duplicate information (if any). For example, a collection client user 130a-n may mistakenly scan the wrong RFID tag for an animal during processing but may not discover the error until after processing was completed. To correct the information, the collection client user 130a-n can search and sort previously collected information to identify the information to be corrected. For example, the collection client user can identify a particular animal, a herd of animals, or all animals processed on a particular date. The collection client processor 124a-n can then provide a suitable graphical user interface for the user to delete or otherwise modify the previously collected information.

In addition, a collection client processor 124a-n can provide a graphical user interface for a user to add information to previously collected information. For example, an animal may be inadvertently missed during processing a group of animals. In such a case, a collection client user 130a-n can add some of all information for the missed animal. Initially, the collection client user 130a-n can identify the animal of interest, an associated herd of animals, or a date on which particular animals were processed. The collection client processor 124a-n can then present a suitable graphical user interface for entry of the missing information.

In one embodiment, a collection client processor 124a-n can provide a graphical user interface to identify duplicate records. In some instances, a collection client user may enter duplicate information, which for example may occur when two or more records indicate the same event was applied to the same animal on the same date but different values are recorded in two different records. If the collection client processor 124a-n identifies two separate but identical records, the collection client processor 124a-n can automatically, or upon user command, eliminate one of the duplicate records. In some instances, if the collection client processor 124a-n identifies duplicate data that conflicts (e.g. two records indicating the same event applied to the same animal on the same date but with different values), the collection client processor 124a-n can display the duplicate information to the collection client user 130a-n, and prompt the user 130a-n to correct the information.

Block 410 is followed by block 412, in which reports can be generated. After animal-related information has been collected and maintained, a collection client user 130a-n can generate at least one report based in part on the collected animal-related data. A report can include, but is not limited to, an animal processing report, an animal statistic report, an animal missing at processing report, an extra animal report, or customized reports. For example, a collection client processor 124a-n can provide a graphical user interface for a collection client user to request a list of animals that were expected to be processed, but for some reason were missing from the processing, i.e. an animal missing at processing report. The collection client processor 124a-n can then determine the selected information, and generate a corresponding report based at least in part on the user's request. In another example, a collection client processor 124a-n can provide a graphical user interface for a collection client user 130a-n to request a list of animals that were not expected to be processed but nonetheless were processed, i.e. an extra animal report. The collection client processor 124a-n can then determine the selected information, and generate a corresponding report based at least in part on the user's request. In the two above examples, a collection client user 130a-n could obtain respective reports to document which data the user may want to add or delete for a particular processing period.

Other reports can be generated in accordance with embodiments of the invention. In one embodiment, a collection client processor 124a-n can provide a graphical user interface for a collection client user 130a-n to provide selections for generating a customized report. Selections can include, but are not limited to, data types, event values, and/or periodic reports, such as the number of animals vaccinated for a particular affliction during the year 2004.

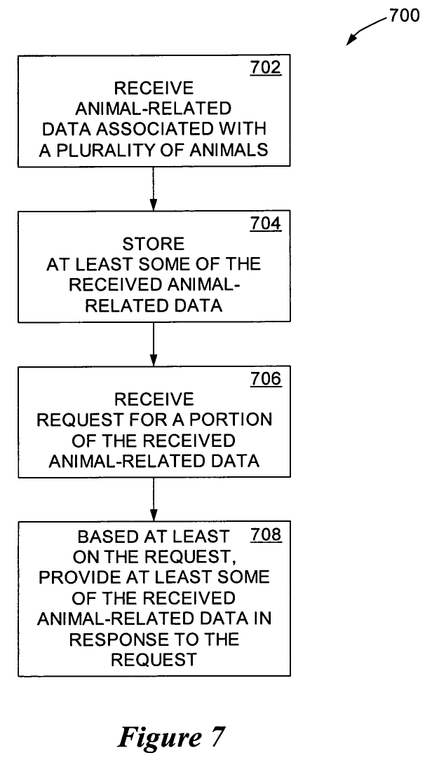

Block 412 is followed by block 414, in which animal-related information is uploaded to a remote server. After animal-related data has been collected and maintained, the collection client processor 124a-n can transmit or otherwise upload the data to a remote server 104. The transmitted or uploaded data can be further processed by the web information system application program 116 and/or stored for subsequent retrieval in memory 120, an associated database such as a web information system database 126, or another suitable data storage device. Further processing performed by the web information system application program 116 is described below in FIG. 7. At block 414, the method 400 ends.

Some or all of the steps of method 400 as described in blocks 402, 404, 406, 408, 410, 412 can be repeated as needed. Other methods in accordance with embodiments of the invention can include some or all of the above steps.

FIG. 5 illustrates another method according to an embodiment of the invention. In the embodiment shown, the method 500 collects and manages animal-related information using a scan card to process one or more animals. The method 500 shown can also be implemented by an EID reader application program, such as 128a-n shown in FIG. 1. In one embodiment, a collection client user 130a-n can operate a client collection device 110a-n, and implement the method 500 described below to collect information related to animals, such as a herd of cattle.

At block 502, the method 500 begins. At block 502, general information is entered. In the embodiment shown, general information associated with a herd of animals can be entered or otherwise received by the EID reader application program 128a-n. The EID reader application program 128a-n can prompt a user 130a-n to enter or can other wise receive general information via a user interface associated with a collection client device 110a-n. The user interface can be a series of webpages or windows adapted to prompt the user 130a-n to enter general information. General information can include, but is not limited to, relevant process name, a working ranch and herd name, destination ranch and herd name, and an animal classification for animals to be processed.

Block 502 is followed by block 504, in which the general information is stored, and the EID reader application program 128a-n is ready to process one or more animals. An individual animal processing card can be utilized to process each animal of a particular herd. An example of a user interface for an individual animal processing card is shown in FIG. 19.

In some instances, an animal's RFID tag or scan card can be lost or broken, and the animal can be retagged. If the animal intended to be retagged can be recognized by other types of identification such a year code, an ear tag number, a Brucellosis tag, or an EID number. If for example, an animal needs to be retagged, then the user interface can provide a button, such as “Retag” button 1902 in FIG. 19, adapted to receive a user input. If the user selects the “Retag” button, the method 500 continues to decision block 506a, and the “YES” branch is followed to decision block 508a.

If, however, no type of identification for the animal is available, then the animal can be retagged as a new animal since no prior history may be available. The user interface can provide another button, such as a “Retag New” button 1904 in FIG. 19. If the user selects the “Retag New” button, the method 500 continues to block 506b, and the “YES” branch is followed to decision block 508b as described in detail below.

Returning to decision block 508a, a determination is made whether an old EID can be located. Using a year code, an ear tag number, a Brucellosis tag, or an EID number, a user 130a-n can input such information into a corresponding user interface or input window associated with the individual animal processing card, such as window 1904 in FIG. 19. The collector client processor 124a-n can utilize such information to locate animal-related information stored in the web information system database 126 or other data source. A list of all animals with similar or matching information can be displayed on an output device associated with the collection client device 110a-n for the user 130a-n to browse or otherwise scroll through. If an EID can be located, then the “YES” branch is followed to block 510a.

In block 510a, the animal is retagged, and the old EID can be replaced with a new EID. The method 500 continues at block 522.

Optionally, the method 500 continues at decision block 512a. At decision block 512a, a determination is made whether to undo data. That is, to undo all events applied to the current animal, and no data will be associated with the current animal. For example, the user interface 1900 in FIG. 19 provides an “Undo Data” button 1906 to implement this function. If a user 130a-n desires to undo data, then the “YES” branch is followed to decision block 514a.

In decision block 514a, a determination is made whether to process the current animal. If a user 130a-n desires to process the current animal, then the “YES” branch is followed to block 522, and the method 500 continues.

Returning to decision block 514a, if a user 130a-n desires not to process the current animal, then the “NO” branch is followed to block 528, and the method 500 continues.

Returning to decision block 512a, if a user 130a-n desires not to undo data, then the “NO” branch is followed to block 524, and the method 500 continues.

Returning to decision block 508a, if an EID cannot be located at block 508a, then the “NO” branch is followed to decision block 506b, and the method 500 continues.

As described above, a user interface can provide another button, such as a “Retag New” button 1904 in FIG. 19. If a user 130a-n selects the “Retag New” button or if an old EID cannot be found at decision block 508a, the method 500 continues to decision block 506b, and the “YES” branch is followed to decision block 508b.

At block 508b, the animal is retagged, and a new EID is assigned to the animal. The method 500 continues at block 522 described below.

Optionally, the method 500 continues at decision block 510b. At decision block 510b, a determination is made whether to undo data. That is, processing of the current animal will cease, and no data will be associated with the current animal. For example, the user interface 1900 in FIG. 19 provides an “Undo Animal” button 1908 to implement this function. If a user 130a-n desires to undo data, then the “YES” branch is followed to decision block 512b.

In decision block 512b, a determination is made whether to process the current animal. If the user desires to process the current animal, then the “YES” branch is followed to decision block 522 described below.

Returning to decision block 512b, if the user desires not to process the current animal, then the “NO” branch is followed to block 528, and the method 500 continues.

Returning to block 504, in many instances, an animal's RFID tag or scan card can be read. In these instances, the method 500 continues to block 506a or block 506b, and the “NO” branch is followed to block 516, in which the animal EID number can be entered or otherwise received via a collection client device 110a-n. As described in FIG. 19, the individual animal processing card 1900 can receive an input such as an animal EID number. If the animal EID number is an existing number and corresponding data associated with a working herd exists, then the corresponding or resulting data can be displayed via the individual animal processing card 1900.

Block 516 is followed by decision block 518, in which a determination is made whether the animal is new. For example, the user can be prompted by a user interface to input whether the animal is new to the ranch. If the animal is new, then the “YES” branch is followed to decision block 520.

At decision block 520, a determination is made whether to process the new animal. For example, the user can be prompted by a user interface to input whether the animal is to be processed. If the new animal is to be processed, then the “YES” branch is followed to block 522 described below.

Returning to decision block 518, if the animal is not new, the method 500 follows the “NO” branch to decision block 522 described below.

Returning to decision block 520, if the animal is not to be processed, then the “NO” branch is followed to block 530 described below.

At decision block 522, a determination is made whether to undo data. That is, processing of the current animal will cease, and no data will be associated with the current animal. If a user 130a-n desires to undo data, then the “YES” branch is followed to block 504 described above and the method 500 continues.

Returning to decision block 522, if a user 130a-n does not desire to undo data, then the “NO” branch is followed to block 524.

At block 524, a scan card EID is entered. The method 500 continues at block 526.

At block 526, event values are input. In the embodiment shown, each event group can be associated with a predefined event value. An event value can include, but is not limited to, a series of characters, numbers, or any combination thereof. For example, an event value can include a prefix portion with five alphanumeric digits, and a numeric portion with four digits.

The method continues at decision block 528.

At decision block 528, a determination is made whether the animal has an animal classification. If a determination is made that the animal has a classification, then the “YES” branch is followed to block 532 described below.

Returning to decision block 528, if a determination is made that the animal does not have a classification, then the “NO” branch is followed to block 530.

At block 530, an animal classification is entered. Using a user interface associated with the individual animal processing card 1900, a user 130a-n can enter an animal classification for the animal.

Block 530 is followed by decision block 532, in which a determination is made whether the animal is known to have a birth date. If a birth date is known, the “NO” branch is followed to block 534.

At block 534, a birth date is entered. Using a user interface associated with the individual animal processing card 1900, a user 130a-n can enter a birth date for the animal.

Returning to decision block 532, If a birth date is not known, then the “YES” branch is followed to block 536. For example. If the birth date is not known, then a default birth date such as the system or processor time can be selected by the collection client processor 124a-n and entered as a birth date.

At block 536, a destination herd is specified. Using a user interface associated with the individual animal processing card 1900, a user 130a-n can enter a destination herd and other associated data for the animal. For example, the collection client processor 124a-n can display via a user interface a selection of available herds to select from. A user 130a-n can scroll through the user interface and select a destination herd from the selection of available herds.

Block 536 is followed by optional block 538, in which single events can be applied. Single events can be applied by the user viewing a user interface such as an individual animal processing card 1900 in FIG. 19. The collection client processor 124a-n can display available events for the particular animal, such as breeding year, breeding season. Year code, breed, birth date, and animal classification. One or more events can be selected for the animal, and applied by the user's selection of the event via the user interface. Previously defined events for a particular animal can be deleted via the user interface as needed.

In one example, the collection client processor 124a-n can display a user interface such as an individual animal processing card 1900 to prompt a user 130a-n to store all previously entered data for subsequent retrieval. If a user 130a-n desires to store the previously entered data, the user 130a-n can indicate the desire via the user interface, and the data can be stored in RAM 122a-n, the web information system database, or other data storage device.

In another example, the collection client processor 124a-n can display a user interface such as an individual animal processing card 1900 with buttons such as “Undo Data” and “Undo Animal.” If for example, a user 130a-n desires to undo all events associated with the current animal and reprocess the animal as necessary, the user 130a-n can select the “Undo Data” button. The method 500 continues to block 540, and the “YES” branch is followed to decision block 542. At decision block 542, a determination is made whether to process the current animal. If the user decides to process the current animal, then the “YES” branch is followed to block 524 described above.

Returning to decision block 542, if the user decides not to process the current animal, then the “NO” branch is followed to block 528 described above.

Returning to optional block 538, if a user 130a-n desires to stop processing the current animal, the user 130a-n can select the “Undo Animal” button. Previously entered data for the current animal will not be saved. The method 500 continues at decision block 544, and the “YES” branch is followed to block 546 described below.

Returning to block 536, in some instances, a user 130a-n can decide not to apply single events, and the method 500 continues through either decision block 540 or decision block 544, and the “NO” branch is followed to block 546.

At block 546, after any previously entered data has been stored or deleted, the next animal can be processed, and the method 500 returns to block 502 described above.

Some or all of the steps of method 500 as described in blocks 502 through 546 can be repeated as needed. Other methods in accordance with embodiments of the invention can include some or all of the above steps.

FIG. 6 illustrates another method according to an embodiment of the invention. In the embodiment shown, the method 600 collects and manages animal-related information using an event group to process one or more animals. The method 600 shown can also be implemented by an EID reader application program, such as 128a-n shown in FIG. 1. In one embodiment, a collection device user 130a-n can operate a client collection device 110a-n, and implement the method 600 described below to collect information related to animals, such as a herd of cattle.

At block 602, the method 600 begins. At block 602, general information is entered. Similar to block 502, in this embodiment, general information associated with a herd of animals can be entered or otherwise received by the EID reader application program 128a-n. The EID reader application program 128a-n can prompt a user 130a-n to enter or can other wise receive general information via a user interface associated with a collection client device 110a-n. The user interface can be a series of webpages or windows adapted to prompt the user 130a-n to enter general information. General information can include, but is not limited to, relevant process name, a working ranch and herd name, destination ranch and herd name, and an animal classification for animals to be processed.

Block 602 is followed by block 604, in which the general information is stored, and the EID reader application program 128a-n is ready to process one or more animals. Similar to block 504, an individual animal processing card can be utilized to process each animal of a particular herd. An example of a user interface for an individual animal processing card is shown in FIG. 19.

In some instances, an animal's RFID tag or scan card can be lost or broken, and the animal can be retagged. If the animal intended to be retagged can be recognized by other types of identification such a year code, an ear tag number, a Brucellosis tag, or an EID number. If for example, an animal needs to be retagged, then the user interface can provide a button, such as “Retag” button 1902 in FIG. 19, adapted to receive a user input. If a user 130a-n selects the “Retag” button, the method 600 continues to decision block 606a, and the “YES” branch is followed to decision block 608a.

If, however, no type of identification for the animal is available, then the animal should be retagged as a new animal since no prior history may be available. The user interface can provide another button, such as a “Retag New” button 1904 in FIG. 19. If the user selects the “Retag New” button, the method 600 continues to block 606b, and the “YES” branch is followed to decision block 608b as described below.

Returning to decision block 608a, a determination is made whether an old EID can be located. Using a year code, an ear tag number, a Brucellosis tag, or an EID number, a user 130a-n can input such information into a corresponding user interface or input window associated with the individual animal processing card, such as window 1904 in FIG. 19. The collector client processor 124a-n can utilize such information to locate animal-related information stored in the web information system database 126 or other data source. A list of all animals with similar or matching information can be displayed on an output device associated with the collection client device 110a-n for the user 130a-n to browse or otherwise scroll through. If an EID can be located, then the “YES” branch is followed to block 610a.

At decision block 610a, the animal is retagged, and the old EID is replaced with a new EID. The method 600 continues at decision block 624 described below.

Optionally, the method 600 continues at decision block 612a. At decision block 612a, a determination is made whether to undo data. That is, to undo all events applied to the current animal, and no data will be associated with the current animal. For example, the user interface 1900 in FIG. 19 provides a “Undo Data” button 1906 to implement this function. If a user 130a-n desires to undo data, then the “YES” branch is followed to decision block 614a.

At decision block 614a, a determination is made whether to process the current animal. If a user 130a-n desires to process the current animal, then the “YES” branch is followed to block 624, and the method 600 continues.

Returning to decision block 614a, if a user 130a-n desires not to process the current animal, then the “NO” branch is followed to block 630 described below, and the method 600 continues.

Returning to decision block 612a, if a user 130a-n desires not to undo data, then the “NO” branch is followed to block 624, and the method 600 continues.

Returning to decision block 608a, if an EID cannot be located at decision block 608a, then the “NO” branch is followed to decision block 608b described below, and the method 600 continues.

As described above, a user interface can provide another button, such as a “Retag New” button 1904 in FIG. 19. If a user 130a-n selects the “Retag New” button or if an old EID cannot be found at decision block 608a, the method 600 continues to decision block 606b, and the “YES” branch is followed to decision block 608b.

At block 608b, the animal is retagged, and a new EID is assigned to the animal. The method 600 continues at block 624.

Optionally, the method 600 continues at decision block 610b. At decision block 610b, a determination is made whether to undo data. That is, processing of the current animal will cease, and no data will be associated with the current animal. For example, the user interface 1900 in FIG. 19 provides a “Undo Animal” button 1908 to implement this function. If a user 130a-n desires to undo data, then the “YES” branch is followed to decision block 612b.

In decision block 612b, a determination is made whether to process the current animal. If a user 130a-n desires to process the current animal, then the “YES” branch is followed to block 624 described below, and the method 600 continues.

Returning to decision block 612b, if a user 130a-n desires not to process the current animal, then the “NO” branch is followed to block 630 described below, and the method 600 continues.

Returning to block 604, in many instances, an animal's RFID tag or scan card can be read. In these instances, the method 600 continues to decision block 606a or decision block 606b, and the “NO” branch is followed to decision block 616, in which a determination is made whether an event group has been selected. As described in FIG. 19, the individual animal processing card 1900 can receive an input such as an event group. One or more event groups can be selected by a user 130a-n, and the corresponding or resulting data can be displayed via the individual animal processing card 1900. Whether an event group has been selected yet, the method 600 can continue at decision block 618.

Decision block 616 is followed by block 618, in which an animal EID is entered. For example, an animal EID can be scanned or otherwise received by the collection client device 110a-n.

Block 618 is followed by decision block 620, in which a determination is made whether the animal is a new animal. For example, an animal EID can be scanned or otherwise received by the collection client device 110a-n, and the user 130a-n can be prompted by a user interface to input whether the animal is new to the ranch. If the animal is new, then the “YES” branch is followed to decision block 622.

At decision block 622, a determination is made whether to process the new animal. For example, a user 130a-n can be prompted by a user interface to input whether the animal is to be processed. If the new animal is to be processed, then the “YES” branch is followed to block 624 described below.

Returning to decision block 620, if the animal is not new, the method 600 follows the “NO” branch to decision block 624 described below.

Returning to decision block 622, if the animal is not to be processed, then the “NO” branch is followed to block 634 described below.

At decision block 624. a determination is made whether to undo data. That is, to undo all events applied to the current animal, and no data will be associated with the current animal. If a user desires to undo data, then the “YES” branch is followed to block 604, described above, and the method 600 continues.

Returning to decision block 624, if the user does not desire to undo data, then the “NO” branch is followed to decision block 626.

At decision block 626, a determination is made whether an event group has been selected. If an event group has been selected, then the “YES” branch is followed to block 630 described below.

If an event group has not been selected, the “NO” branch is followed to block 628. At block 628, an event group is selected. In this embodiment, an event group can be selected by a user 130a-n, or a default or automatic selection can be made by the collection client processor 124a-n.

Block 628 is followed by block 630, in which an event value is input. In the embodiment shown, each event group can be associated with a predefined event value. An event value can include, but is not limited to, a series of characters, numbers, or any combination thereof. For example, an event value can include a prefix portion with five alphanumeric digits, and a numeric portion with four digits.

Block 630 is followed by decision block 632, in which a determination is made whether the animal has an animal classification. If a determination is made that the animal has a classification, then the “YES” branch is followed to decision block 636 described below.

Returning to decision block 632, if a determination is made that the animal does not have a classification, then the “NO” branch is followed to block 634.

At block 634, an animal classification is entered. Using a user interface associated with the individual animal processing card 1900, a user 130a-n can enter an animal classification for the animal.

Block 634 is followed by decision block 636, in which a determination is made whether the animal is known to have a birth date. If a birth date is known, the “NO” branch is followed to block 638.

At block 638, a birth date is entered. Using a user interface associated with the individual animal processing card 1900, a user 130a-n can enter a birth date for the animal.

Returning to decision block 636, If a birth date is not known, then the “YES” branch is followed to block 640 described below. For example. If the birth date is not known, then a default birth date such as the system or processor time can be selected by the collection client processor 124a-n and entered as a birth date.

At block 640, a destination herd is specified. Using a user interface associated with the individual animal processing card 1900, a user 130a-n can enter a destination herd and other associated data for the animal. For example, the collection client processor 124a-n can display via a user interface a selection of available herds to select from. The user 130a-n can scroll through the user interface and select a destination herd from the selection of available herds.

Block 640 is followed by optional block 642, in which single events can be applied. Single events can be applied by the user viewing a user interface such as an individual animal processing card 1900 in FIG. 19. The collection client processor 124a-n can display available events for the particular animal, such as breeding year, breeding season. Year code, breed, birth date, and animal classification. One or more events can be selected for the animal, and applied by the user's selection of the event via the user interface. Previously defined events for a particular animal can be deleted via the user interface as needed.

In one example, the collection client processor 124a-n can display a user interface such as an individual animal processing card 1900 to prompt a user 130a-n to store all previously entered data for subsequent retrieval. If a user 130a-n desires to store the previously entered data, the user can indicate the desire via the user interface, and the data can be stored in RAM 122a-n, the web information system database, or other data storage device.

In another example, the collection client processor 124a-n can display a user interface such as an individual animal processing card 1900 with buttons such as “Undo Data” and “Undo Animal.” If for example, a user 130a-n desires to undo all events associated with the current animal and reprocess the animal as necessary, the user 130a-n can select the “Undo Data” button. The method 600 continues to decision block 644, and the “YES” branch is followed to decision block 646. At decision block 646, a determination is made whether to process the current animal. If a user 130a-n decides to process the current animal, then the “YES” branch is followed to block 528 described above, where the method 600 continues.

Returning to decision block 646, if the user 130a-n decides not to process the current animal, then the “NO” branch is followed to block 632 described above, and the method 600 continues.

Returning to optional block 642, if a user 130a-n desires to stop processing the current animal, the user 130a-n can select the “Undo Animal” button. Previously entered data for the current animal will not be saved for subsequent retrieval. The method 600 continues at decision block 648, and the “YES” branch is followed to block 650 described below.

Returning to block 642, in some instances, a user 130a-n can decide not to apply single events, and the method 600 continues through either decision block 644 or decision block 648, and the “NO” branch is followed to block 650.

At block 650, after any previously entered data has been stored or deleted, the next animal can be processed, and the method 600 returns to block 602 described above.

Some or all of the steps of method 600 as described in blocks 602 through 650 can be repeated as needed. Other methods in accordance with embodiments of the invention can include some or all of the above steps.

B. Data Management

FIG. 7 illustrates a method according to another embodiment of the invention. In the embodiment shown, the method 700 provides access and management functionality for cattle-related information. The method 700 shown can be implemented by a web information system application program, such as 116 shown in FIG. 1. In one embodiment, the method 700 can also be used to implement some or all functional modules associated with a web information system application program 116, such as cattle management, semen management, medicine management, pasture management, payment management, and feedlot data uploading.

The method 700 begins at 702, in which animal-related data is received. In the embodiment shown in FIG. 7, animal-related data can be received from a collection client device 110a-n. The collection client device 110a-n can initially receive and process animal-related data, such as during the processing of one or more animals as described above in method 400 of FIG. 4. After an animal has been processed, the collection client device 110a-n can upload the information to the server 104 for further processing.

Block 702 is followed by block 704, in which some or all of the received animal-related data is stored. In the embodiment shown in FIG. 7, some or all of the received animal-related data can be stored in memory 120, a web information system database 126, or another data storage device.

Block 704 is followed by block 706, in which a request for a portion of the animal-related data is received. In the embodiment shown in FIG. 7, a request from an access client device 102a-n or a collection client device 110a-n can be received by the server 104. The request can include instructions from a user 108a-n, 130a-n or can be automatically generated instructions requesting some or all of the animal-data stored in memory 120, a web information system database 126, or another data storage device.

In one embodiment, an access client device user 108a-n can operate an access client device 102a-n and transmit a request via the network 106 to the server 104. Through a graphical user interface, such as a web browser application program, the user 108a-n can communicate with the server 104 and utilize functionality of the web information system application program 116. For example, a website associated with the web information system application program 116 can provide a request function for the user 108a-n to select. The user 108a-n can then generate and transmit a request by selecting the corresponding request function. A request can include, but is not limited to, a selection of an icon, selection of a menu item, data input, a selection of an instruction, selection by an data input device, or a request for a customized report.

Block 706 is followed by block 708, in which a portion of the previously stored data is provided based at least in part on the request. In the embodiment shown in FIG. 7, the server processor 118 can determine a selection of data based at least in part on the request. The server processor 118 can then provide the selected data by transmitting the data to the source of the request, such as an access client device 102a-n or to a collection client device 110a-n. The method 700 ends at block 708.

Some or all of the steps of method 700 as described in blocks 702, 704, 706, and 708 can be repeated as needed. Other methods in accordance with embodiments of the invention can include some or all of the above steps.

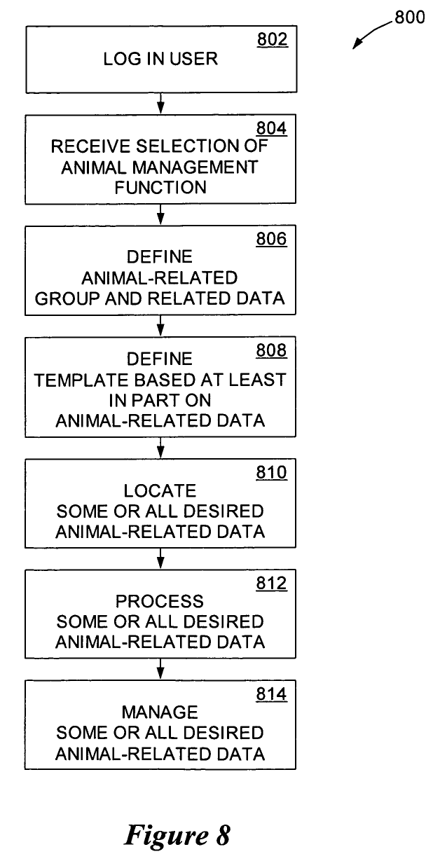

FIG. 8 illustrates another method according to another embodiment of the invention. In the embodiment shown, the method 800 provides access and management functionality for cattle-related information. The method 800 shown can be implemented by a web information system application program, such as 116 shown in FIG. 1. In one embodiment, the method 800 can also be used to implement some or all functional modules associated with a web information system application program 116, such as cattle management, semen management, medicine management, pasture management, payment management, and feedlot data uploading. For example, an access client device user 108a-n can access the web information system application program 116 via an access client device 102a. The user 108-an can interact with the functionality provided by the web information system application program 116 via a network 106 such as the Internet.

The method 800 begins at block 802, in which a user logs in via a network. In the embodiment shown in FIG. 8, an access client user 108a-n or a collection client user 130a-n can access a web information system application program 116.

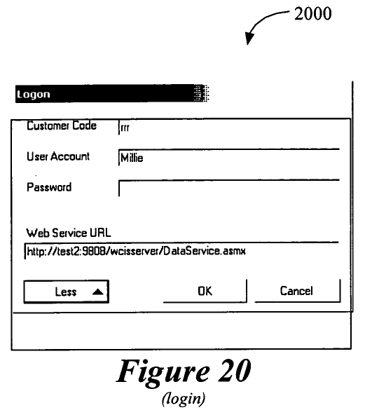

In one embodiment, an access client device user 108a-n can operate an access client device 102a-n and communicate via the network 106 with the server 104. Through a graphical user interface, such as a web browser application program, the user can communicate with the server 104 and utilize functionality of the web information system application program 116. Prior to permitting access to some or all of the functionality, the server 104 can utilize an authentication method or device to log in or otherwise verify the identity of the access client site user 108a-n. An example of a user interface to log in a user is shown as 2000 in FIG. 20, and is further described below. After the user 108a-n is authenticated or his/her identity is otherwise verified, the server 104 can permit the user 108a-n access functionality associated with the web information system application program 116, such as sending a request for some or all of the data in memory 120, the web information system database, or another data storage device.

Block 802 is followed by block 804, in which an animal management function is selected. In the embodiment shown in FIG. 8, an access client user 108a-n or a collection client user 130a-n can select one or more animal management functions displayed on a website associated with the web information system application program 116.

In one embodiment, after the access site user 108a-n has logged in, the user 108a-n can access various functionality associated with a web information system application program such cattle management, semen management, medicine management, pasture management, payment management, and feedlot data.

For example, a graphical user interface such as a website associated with the web information system application program can provide an access client site user 108a-n access to various functionality associated with cattle management. The user 108a-n can utilize the website and various associated input devices to perform cattle management functions including, but not limited to, setting up a ranch, herd and/or group information, setting up templates for processing animal-related data, preparing for processing a desired set of animals according to a desired processing regimen, processing animals to collect and store the desired data, verifying/maintaining the desired data, generating/managing reports, and uploading data to a web information system database 126. The website can provide a series of display screens that can present information and data to the user 108a-n in various convenient and informative formats. Examples of display screens are shown in FIGS. 20-35.

In another example, a graphical user interface such as a website associated with the web information system application program can provide an access client site user 108a-n access to various functionality associated with semen management. The user 108a-n can utilize the website and various associated input devices to perform semen management functions including, but not limited to, initialize semen inventory, record purchases and sales of semen, check out and check in semen for inventory, check inventory, adjust inventory, generate a weekly semen inventory report, or customize a report upon request.

In yet another example, a graphical user interface such as a website associated with the web information system application program can provide an access client site user 108a-n access to various functionality associated with medicine management. The user 108a-n can utilize the website and various associated input devices to perform medicine management functions including, but not limited to, initialize medicine inventory, record purchases of medicines, check out and check in medicines before and after processing, check medicine inventory, adjust medicine inventory, generate a weekly medicine inventory report, or generate a customized report.

By way of another example, a graphical user interface such as a website associated with the web information system application program can provide an access client site user 108a-n access to various functionality associated with pasture management. The user 108a-n can utilize the website and various associated input devices to perform pasture management functions including, but not limited to, add a new pasture, edit a pasture, delete a pasture, close a pasture, make a pasture rotation schedule, view/edit a pasture rotation schedule, delete a pasture rotation schedule, record an actual herd movement, view/edit an actual herd movement, delete an actual herd movement, check pasture rotation status, generate a pasture rotation report, or generate a customized report.

By way of another example, a graphical user interface such as a website associated with the web information system application program can provide an access client site user 108a-n access to various functionality associated with payment management. The user 108a-n can utilize the website and various associated input devices to perform payment management functions including, but not limited to, manage user accounts, and manage payments including payment amounts, fees, stop service, and reminders.

In another example, a graphical user interface such as a website associated with the web information system application program can provide an access client site user 108a-n access to various functionality associated with feedlot data uploading management. The user 108a-n can utilize the website and various associated input devices to perform feedlot data uploading management functions including, but not limited to, uploading feedlot data, and monitoring data uploading.

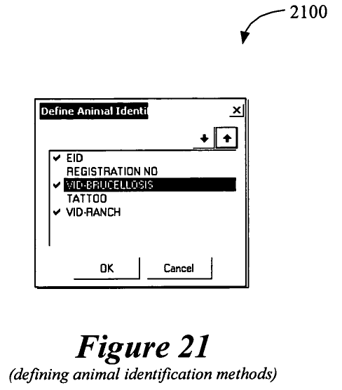

In one embodiment, the web information system application program 116 can permit a user 108a-n, 130a-n to define an animal identification method. Such identification methods can be used by the web information system application program 116 to refer to and uniquely identify specific animals within database records. An example of a user interface to permit a user 108a-n to define one or more animal identification methods is shown as 2100 in FIG. 21 and described below. Various identification methods and techniques can be used including, but not limited to, an EID, a pure breed registration number, a tattoo, a Brucellosis tag (VID-Brucellosis), and an ear tag (VID-Ranch). One or more identification methods and techniques can be defined by a user via a user interface associated with the web information system application program 116. User selections of animal identification methods and techniques can be stored by the web information system application program 116 for subsequent retrieval and reference.

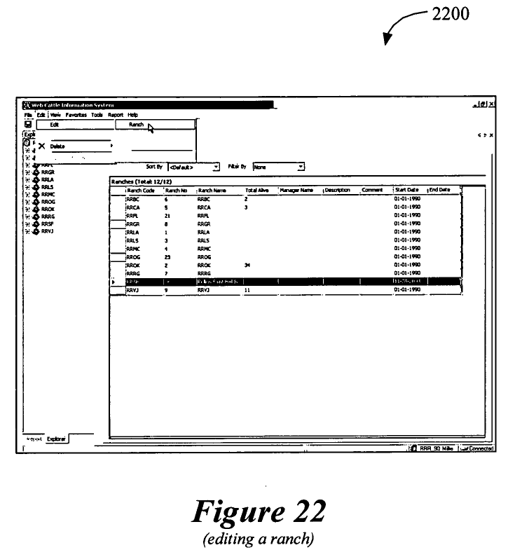

Block 804 is followed by block 806, in which a group based in part on at least animal-related information is defined. In the embodiment shown in FIG. 8, a user can define an animal-related group such as setting up a ranch. Using a graphical user interface such as a website associated with the web information system application program, the user 108a-n can determine whether data for an animal-related group, such as a ranch, has been stored. If a particular ranch exists, the user can select the desired ranch or other animal-related group to manage. Related information to the ranch or other animal-related group can be further accessed. An example of a user interface to select a ranch for editing is shown as 2200 in FIG. 22 described below.

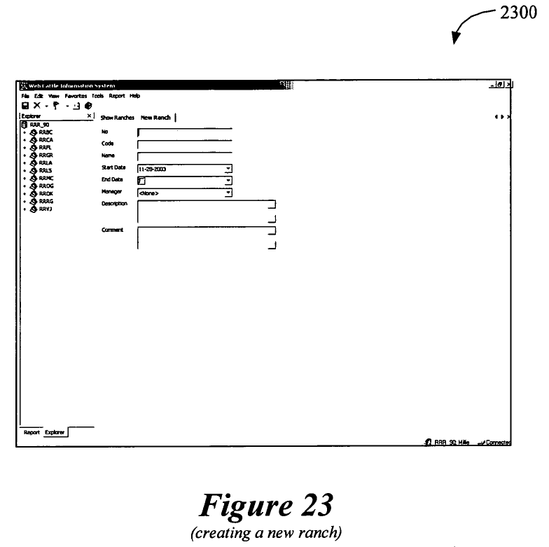

If a particular animal-related group does not exist, then via the graphical user interface the user 108a-n can instruct the system to define a new ranch. An example of a user interface to define a new ranch is shown as 2300 in FIG. 23 and described below. The graphical user interface can provide data input windows or other input devices for the user to define a new ranch, new herds within that ranch, new groups within the ranch, a ranch number, a ranch name, a date the ranch was put into use, a name of the ranch manager, and a description of the ranch.

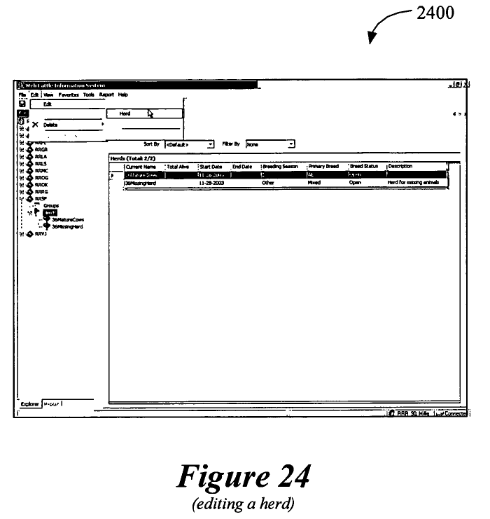

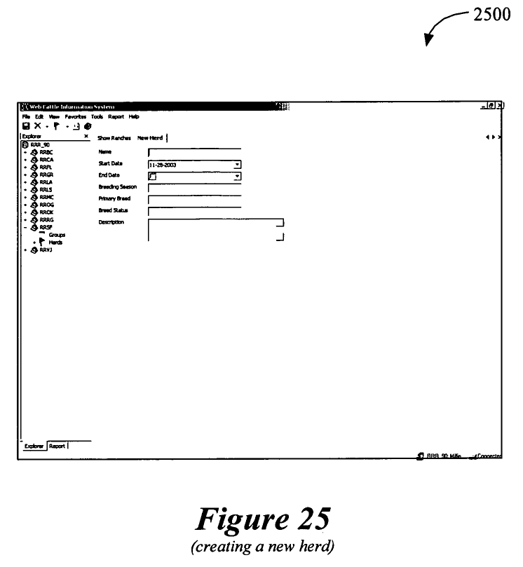

After defining a new ranch, the access site user 108a-n can then define herds within a particular ranch. The graphical user interface can display a window that lists all of the available herds for the particular ranch. An example of a user interface to select a herd for editing is shown as 2400 in FIG. 24 described below. If the herd of interest is not available, the access site user 108a-n can then define a new herd. Another graphical user interface can provide data input windows or other input devices for the user to define a new herd, a herd name, a date the herd was put into use, a breeding season for the herd, a primary breed of the animals in the herd, a breeding status of the herd, and a description of the herd. An example of a user interface to create a new herd is shown as 2500 in FIG. 25 described below.

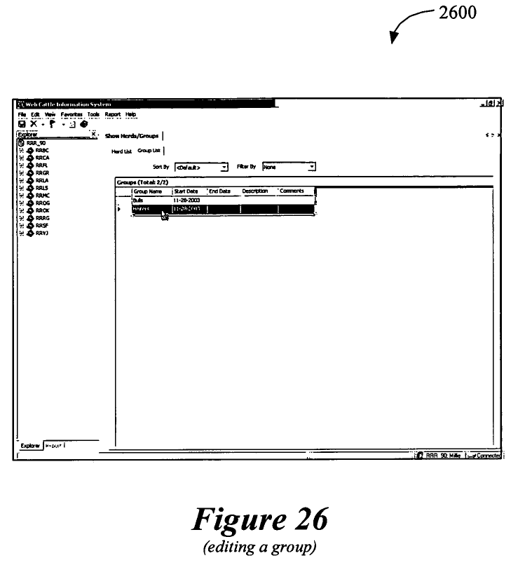

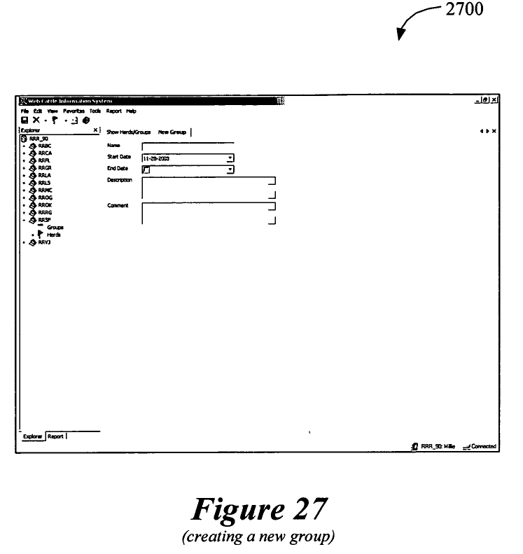

In addition to defining ranches and herds, the access site user 108a-n can also define groups of interest. The graphical user interface can display a window that lists all of the available groups for a particular ranch. An example of a user interface for selecting a group of interest for editing is shown as 2600 in FIG. 26 described below. If the group of interest is not available, the access site user 108a-n can then define a new group. Another graphical user interface can provide data input windows or other input devices for the user to define a group, a group name, a date the group was put into use, and a description of the group. An example of a user interface for creating a new group is shown as 2700 in FIG. 27 described below.

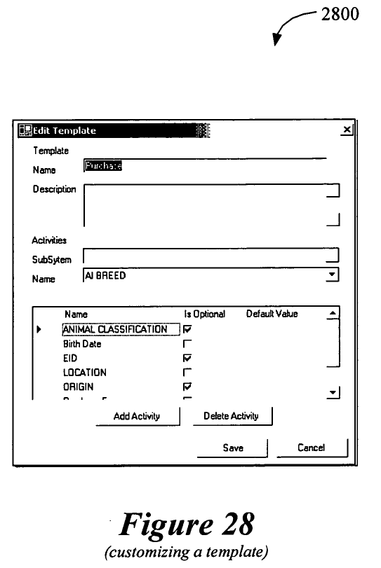

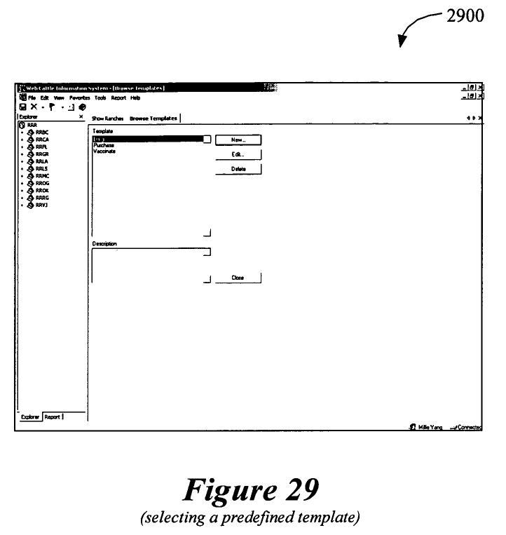

Block 806 is followed by block 808, in which a template based in part on the animal-related information is defined. In the embodiment shown in FIG. 8, the user can define a new or edit a predefined template based in part on at least animal-related information, such as a ranch. An example of a user interface for editing and/or defining a template is shown as 2800 in FIG. 28 descrbied below. The access site user 108a-n, for example, can also retrieve previously stored templates. An example of a user interface to select a predefined template for editing is shown as 2900 in FIG. 29 described below. A template can emulate a business process for a location such as a ranch by implementing or otherwise facilitating a series of animal management activities. Templates can be useful for initiating or otherwise continuing a consistent pattern of data entry to regulate the animal data processing. Templates can be implemented for processes such as, but not limited to, handling an animal that are no longer part of the ranch, transferring an animal, documenting the death of an animal, or selling an animal.

For example, an access site user 108a-n can utilize a graphical user interface associated with a web information system application program to generate a template for transferring an animal. The graphical user interface can present a data entry window for the access site user 108a-n to define or otherwise select a predefined template. If no predefined or desired template exists, the access site user 108a-n can enter a template name, description of the template, and associate one or more activities and/or activity codes with the template. In this example, the access site user 108a-n can associate four activity codes with the template, such as EID, location, herd, and transfer. Other activities and/or activity codes can be utilized in accordance with embodiments of the invention. The access site user 108a-n can enter default values for each of the activities as well as a designation of whether a particular activity is optional. Once the template is defined, the template can be stored in memory 120, the web information system database 126, or another suitable data storage device for subsequent retrieval.

Block 808 is followed by block 810, in which some or all of the desired animal-related data is located. In the embodiment shown in FIG. 8, the user can define a search for searching previously collected animal-related data. For example, an access site user 108a-n can search for and view a list of animals of interest. A listing of animals can comprise hundreds or thousands of animals. The access site user 108a-n can utilize a graphical user interface window associated with the web information system application program 116 to select a search filter or to define a search query to locate a particular animal. One or more predefined search filters can be utilized or otherwise modified to locate one or more animals of interest. For example, search filters can include, but are not limited to characteristics such as animal class, animal name, animal status, bangs number, birth date, breeding status, ear tag number, EID, herd name, origin, primary breed, ranch, registration number, sex, year code, or other user-defined filters or criteria. Upon receiving a search query or search filter, the server processor 118 can search data stored in memory, a web information system database 126, or another suitable data storage device for animals that satisfy defined search criteria of the search query and/or search filter. The server processor 118 can then display a listing of any animals satisfying the search criteria.

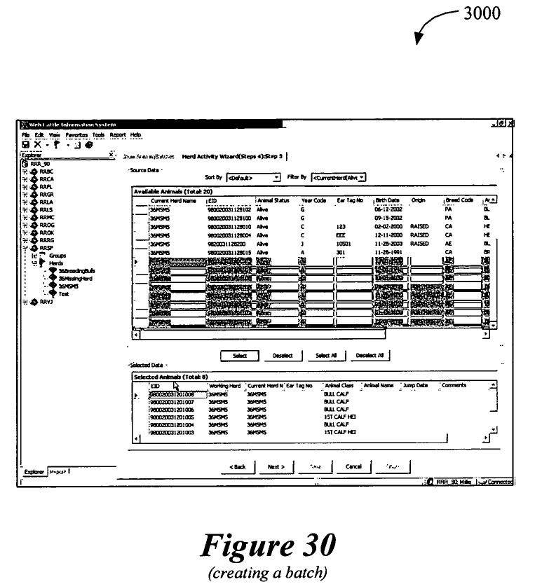

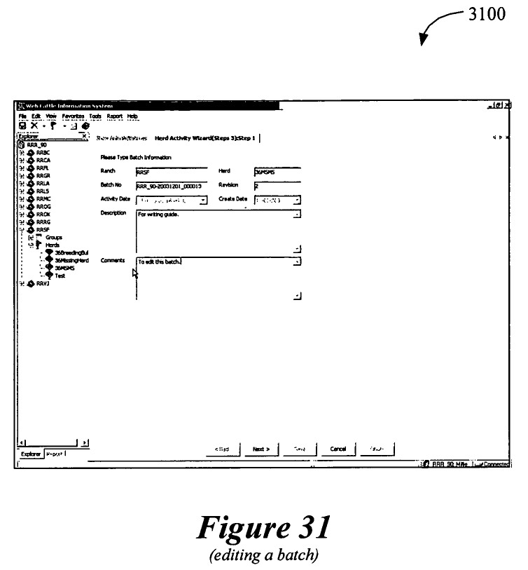

In one embodiment, a user 108a-n can define a batch to locate specific animal-related data. Data used to define a batch can include, but is not limited to, a ranch name or code, a herd name or code, a batch number, a batch revision number, an activity date, a create date, a description, and comments. One or more batches can also be predefined by the system or a user 108a-n and automatically identified by the web information system application program 116 for subsequent retrieval. For example, animal-related data uploaded from an EID reader application program 128a-n, related to a specific herd and processed in a single day, can be automatically defined as a batch. If, for example, processing of a particular herd lasts a week, seven batches can be correspondingly generated after the animal-related data is uploaded from the EID reader application program 128a-n. Each batch can be uniquely identified by a number and a batch revision number to track the revision history of each batch. In this manner, users 108a-n, 130a-n can trace and/or locate processed data. An example of a user interface for creating a batch for animal-related data is shown as 3000 in FIG. 30 described below. An example of a user interface for editing a batch for animal-related data is shown as 3100 in FIG. 31 described below.

Block 810 is followed by block 812, in which some or all of the desired animal-related data is processed. In the embodiment shown in FIG. 8, a user can process some or all of the desired animal-related data. From the above example, after at least one animal of interest is located, an access client user 108a-n can process the respective animal-related data. Because of the dynamic nature of livestock activities, the data collected and uploaded to the remote server for any particular ranch, herd, or group can be monitored and modified as necessary to insure that it reflects the current status of the situation at the ranch. For example, after data for a particular herd has been uploaded to the remote server, a user 108a-n might discover that an animal in the herd has died. To insure that previously collected data is relatively accurate and current, the access site user 108a-n can process the uploaded data to reflect the recent death of the particular animal. Processing can be handled in either by batch or based on activity.

In one embodiment, data can be processed through use of a batch request. As explained above, an access site user 108a-n can define a particular batch of interest to be processed. The server processor 118 can assign a batch number and batch revision number to the request and can present one or more user interface windows that permit the access client user 108a-n to define the particular batch processing request of interest. The access site user 108a-n can designate, for example, a date that activities relevant to the processing request were performed, comments or descriptions of the request for later reference, a particular template, animals to be processed according to the selected template, and any other information associated with a template. Upon completing the request, the batch request can be scheduled for processing and the status of the processing can be monitored by the processor 118. Via a graphical user interface, the access client user 108a-n can monitor the status of the batch processing request to ascertain if and when the processing is completed.

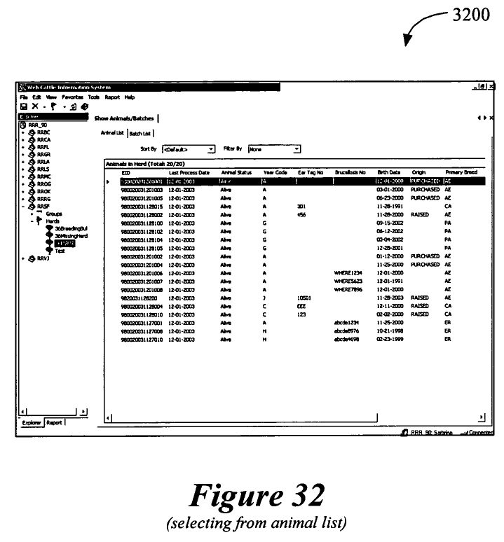

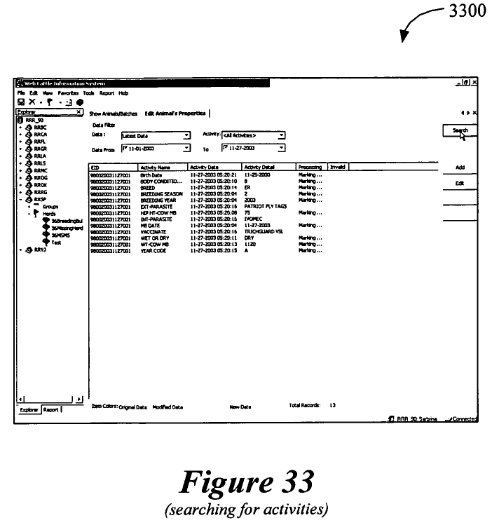

In addition to processing data in batches, an access client user 108a-n can also process data for a specific animal or specific set of animals by adding one or more activities to the data for that animal or animals. To do this, the access site user can first locate the animals of interest. This can be accomplished by searching for the animals in accordance with the approach discussed above. An example of a user interface adapted to search and select a particular animal or set of animals is shown as 3200 in FIG. 32 described below. Once the animal of interest has been located, a graphical user interface window can display animal-related information and can provide functionality for an access client user 108a-n to add one or more activities to the data for the particular animal. In one example, an access client user 108a-n can add an activity and then can select a desired activity from a list of available activities. One or more data entry windows for entering one or more values pertaining to the selected activity can be displayed for the access client user 108a-n. An example of a user interface adapted to search for and edit particular activities is shown as 3300 in FIG. 33 described below. After the access client user 108a-n enters the desired values and adds the desired activities, a batch request can be scheduled for processing and the status of the processing can be monitored. Via a graphical user interface, the access client user 108a-n can monitor the status of the batch processing request to ascertain if and when the processing is completed.

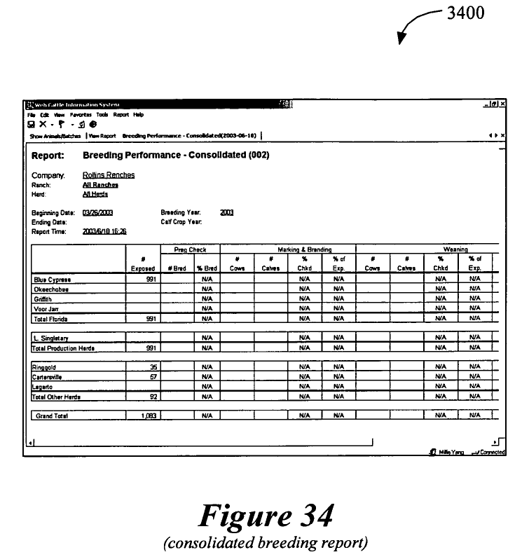

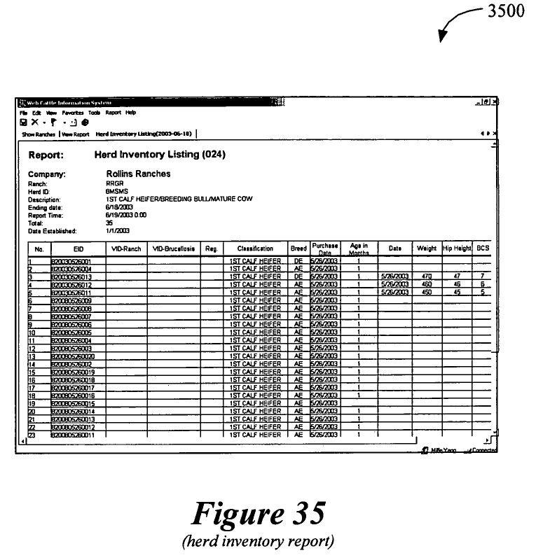

Block 812 is followed by block 814, in which a report based at least in part on the desired animal-related data is generated. In the embodiment shown in FIG. 8, a user can generate, view, download, archive, and generally manage reports based on the desired animal-related data. For example, an access client user 108a-n may want to generate one or more reports summarizing desired animal-related data at a company level related to breeding performance, trend of calves lost, livestock purchases, livestock sales, herd inventory by age, herd inventory by breed, registration status, and transfers. An example of a user interface providing a report showing breeding performance is shown as 3400 in FIG. 34 described below. By way of another example, an access client user 108a-n may want to generate one or more reports based on the desired animal-related data pertaining to a particular company, ranch, herd, or animal. An example of a user interface providing a report showing a herd inventory is shown as 3500 in FIG. 35 described below. In any instance, the web information system application program can generate a report based in part on at least the desired animal-related data. Reports can be scheduled for generation, and the status can be monitored by server processor 118. A list of finished reports and a list of requested but unfinished reports can be displayed via a graphical user interface for monitoring by the access client user 108a-n. Any number of reports can be generated in various formats, electronic, print, file, or any other suitable medium as desired. In at least one embodiment, a report can be transmitted to an access client device 102a-n and/or a collection client device 110a-n. Reports can also be archived in memory, the web information system database 116, or another suitable data storage device for subsequent access and use. Lists of current reports can be updated as needed, and out-of-date reports can be deleted as necessary.

Some or all of the steps of method 800 as described in blocks 802 through 814 can be repeated as needed. Other methods in accordance with embodiments of the invention can include some or all of the above steps.

FIGS. 9-18 illustrate examples of graphical user interface screens for a method in accordance with an embodiment of the invention. The graphical user interface screens shown in FIG. 9-18, can be implemented with a method such as method 400 in FIG. 400 for an EID reader application program shown as 128a-n in FIG. 1. Other graphical user interfaces can be used with other embodiments of the invention, and the screens illustrated as follows are not intended to be limiting.

FIG. 9 illustrates a login window 900 for authenticating a user, or otherwise verifying the identity of a user. Associated data windows for the login window 900 can include, but are not limited to, customer code, user account, password, and web service uniform resource locator (URL). Fewer or greater, and other data windows can be used in accordance with embodiments of the invention.

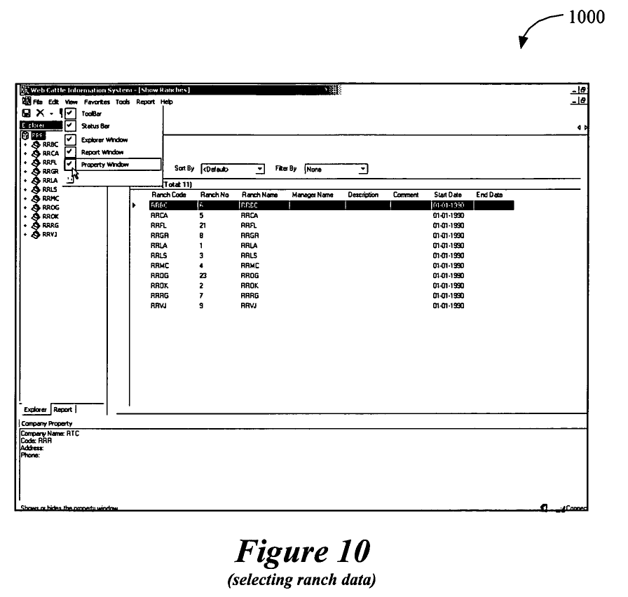

FIG. 10 illustrates a window 1000 for selecting a portion of previously stored animal-related data, such as ranch data. Associated data windows for the window 1000 can include, but are not limited to, ranch code, ranch number, ranch name, manager name, description, comment, start date, and end date. Fewer or greater, and other data windows can be used in accordance with embodiments of the invention.

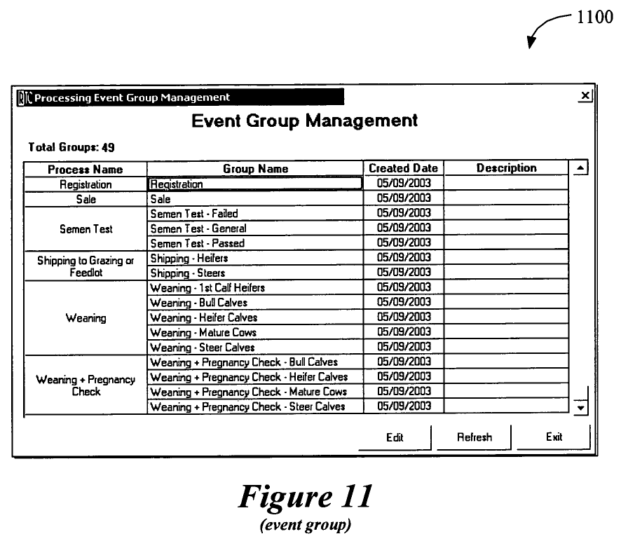

FIG. 11 illustrates a window 1100 for generating a data structure, such as an event group, adapted to be associated with new animal-related data. Associated data windows for the window 1100 can include, but are not limited to, process name, group name, created date, and description. Fewer or greater, and other data windows can be used in accordance with embodiments of the invention.

FIG. 12 illustrates a window 1200 for generating a data structure, such as a scan card, adapted to be associated with new animal-related data. Associated data windows for the window 1200 can include, but are not limited to, EID for scan card, card type, select event group, event detail, and event group. Fewer or greater, and other data windows can be used in accordance with embodiments of the invention.

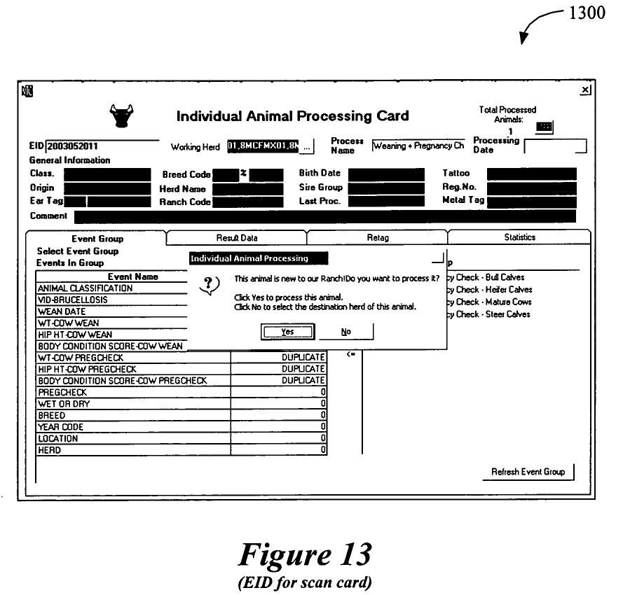

FIG. 13 illustrates a window 1300 for processing an animal and receiving an identifier associated with an animal, wherein the identifier is associated with a scan card. Associated data windows can be adapted to receive new animal-related data for association with the data structure, such as the scan card. Associated data windows for the window 1300 can include, but are not limited to, EID, working herd, process name, processing date, class, breed code, birth date, tattoo, origin, herd name, sire group, registration number, ear tag, ranch code, last processed, metal tag, comment, event group, result data, retag, and statistics. Fewer or greater, and other data windows can be used in accordance with embodiments of the invention.

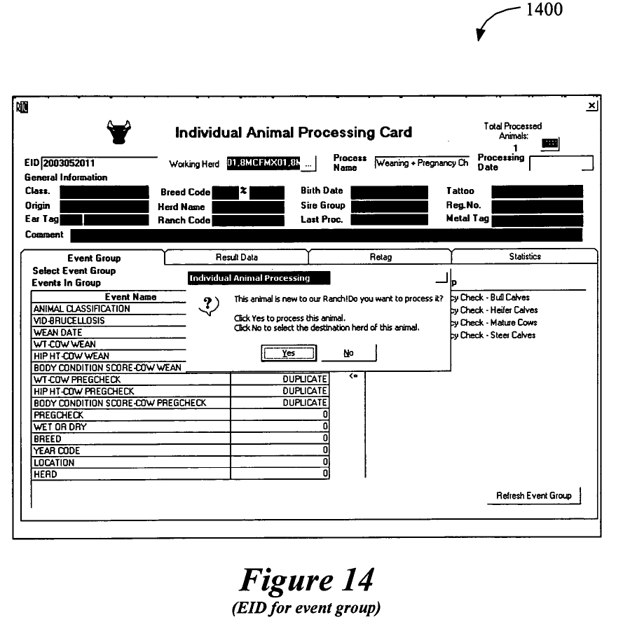

FIG. 14 illustrates a window 1400 for processing an animal and receiving an identifier associated with an animal, wherein the identifier is associated with an event group. Associated data windows can be adapted to receive new animal-related data for association with the data structure, such as the event group. Associated data windows for the window 1400 can include, but are not limited to, EID, working herd, process name, processing date, class, breed code, birth date, tattoo, origin, herd name, sire group, registration number, ear tag, ranch code, last processed, metal tag, comment, event group, result data, retag, and statistics. Fewer or greater, and other data windows can be used in accordance with embodiments of the invention.

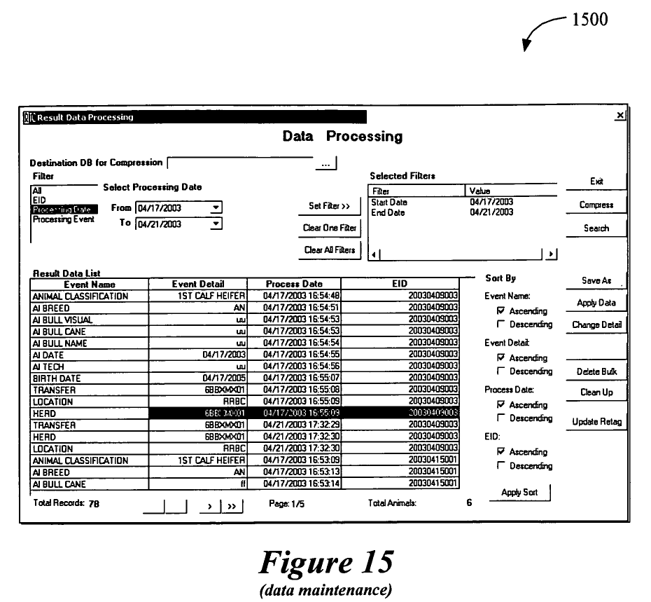

FIG. 15 illustrates a window 1500 for processing animal-related data, including selecting one or more search filters or criteria. Associated data windows for the window 1500 can include, but are not limited to, filter, select processing date, selected filters, result data list, event name, event detail, process date, EID, and “sort by” functionality. Fewer or greater, and other data windows can be used in accordance with embodiments of the invention.

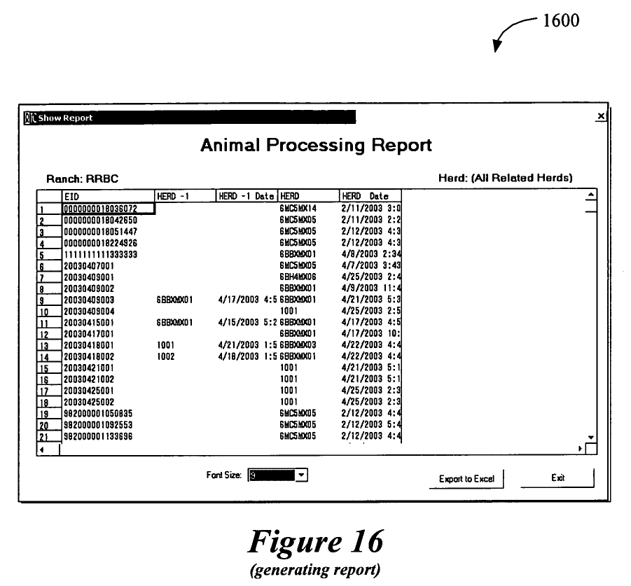

FIG. 16 illustrates a window 1600 with a generated report such as an “animal processing report.” Associated data windows for the window 1600 can include, but are not limited to, ranch, herd (all related herds), EID, herd—1, herd—1 date, herd, and herd date. Fewer or greater, and other data windows can be used in accordance with embodiments of the invention.

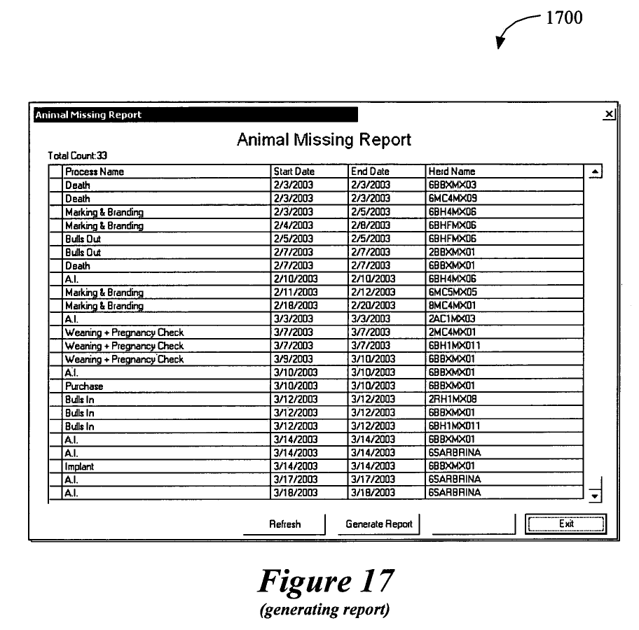

FIG. 17 illustrates a window 1700 with a generated report such as an “animal missing report.” Associated data windows for the window 1700 can include, but are not limited to, total count, process name, start date, end date, and herd name. Fewer or greater, and other data windows can be used in accordance with embodiments of the invention.

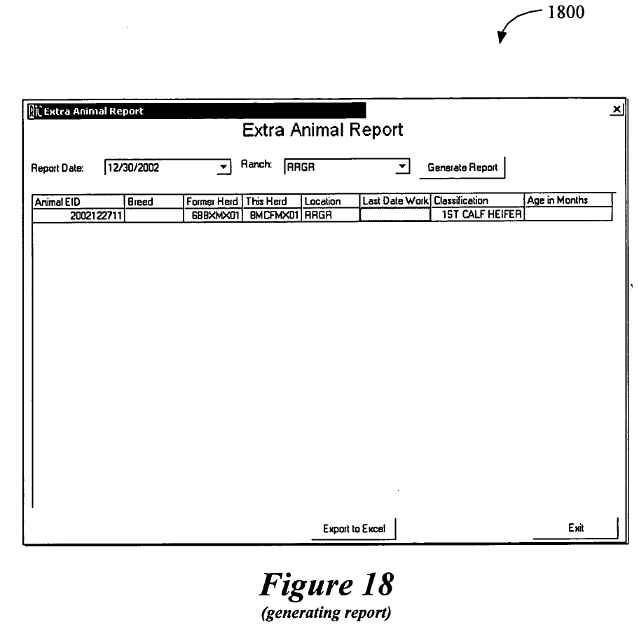

FIG. 18 illustrates a window 1800 with a generated report such as an “extra or missing report.” Associated data windows for the window 1800 can include, but are not limited to, report date, ranch, animal EID, breed, former herd, this herd, location, last date work, classification, and age in months. Fewer or greater, and other data windows can be used in accordance with embodiments of the invention.

FIG. 19 illustrates a window 1900 for collecting and managing animal-related data, including animal-related information using a scan card. Associated data windows for the window 1900 can include, but are not limited to, Retag 1902, Retag New 1904, Pregcheck History Data, General Information, classification, origin, ear tag, Comment, breed code, herd ID, ranch code, birth date, sire group, last processing, tattoo, registration number, metal tag, event group, result data, retag, statistics, current processing details, history processing details, available events, detail. apply, Undo Data 1906, and Undo Animal 1908. Fewer or greater, and other data windows can be used in accordance with embodiments of the invention. Other graphical user interfaces can be used with other embodiments of the invention, and the screen illustrated as follows is not intended to be limiting.

FIGS. 20-35 illustrate examples of graphical user interface screens for a method in accordance with an embodiment of the invention. The graphical user interface screens shown in FIGS. 20-35, can be implemented with a method such as methods 700 and 800 for a web information system application program shown as 116 in FIG. 1. Other graphical user interfaces can be used with other embodiments of the invention, and the screens illustrated as follows are not intended to be limiting.

FIG. 20 illustrates a window 2000 for managing animal-related data, including logging in or otherwise verifying the identity of an access client site user 108a-n. Associated data windows for the window 2000 can include, but are not limited to, customer code, user account, web service URL (uniform resource locator) and a password. Fewer or greater, and other data windows can be used in accordance with embodiments of the invention.

FIG. 21 illustrates a window 2100 for managing animal-related data, including permitting an access client site user 108a-n to define an animal identification method. Associated data windows for the window 2100 can include animal identification methods such as EID, pure breed registration number, tattoo, Brucellosis tag (VID-Brucellosis), and ear tag (VID-Ranch). Fewer or greater, and other data windows can be used in accordance with embodiments of the invention.

FIG. 22 illustrates a window 2200 for managing animal-related data, including editing a ranch template. Associated data windows for the window 2200 can include, but are not limited to, ranch code, ranch number, total alive, manager name, description, comment, start date, and end date. Fewer or greater, and other data windows can be used in accordance with embodiments of the invention.

FIG. 23 illustrates a window 2300 for managing animal-related data, including creating a new ranch template. Associated data windows for the window 2300 can include, but are not limited to, ranch code, ranch number, total alive, manager name, description, comment, start date, and end date. Fewer or greater, and other data windows can be used in accordance with embodiments of the invention.

FIG. 24 illustrates a window 2400 for managing animal-related data, including editing a herd template. Associated data windows for the window 2400 can include, but are not limited to, total alive, start date, end date, breeding season, primary breed, breed status, and description. Fewer or greater, and other data windows can be used in accordance with embodiments of the invention.

FIG. 25 illustrates a window 2500 for managing animal-related data, including creating a new herd template. Associated data windows for the window 2500 can include, but are not limited to, total alive, start date, end date, breeding season, primary breed, breed status, and description. Fewer or greater, and other data windows can be used in accordance with embodiments of the invention.

FIG. 26 illustrates a window 2600 for managing animal-related data, including editing a group template. Associated data windows for the window 2600 can include, but are not limited to, a group name, start date, end date, description, and comments. Fewer or greater, and other data windows can be used in accordance with embodiments of the invention.

FIG. 27 illustrates a window 2700 for managing animal-related data, including creating a new group template. Associated data windows for the window 2700 can include, but are not limited to, a group name, start date, end date, description, and comments. Fewer or greater, and other data windows can be used in accordance with embodiments of the invention.

FIG. 28 illustrates a window 2800 for managing animal-related data, including customizing a template. Associated data windows for the window 2800 can include, but are not limited to, template name, template description, subsystem activities, activity names such as AI Breed, Animal Classification, Birth Date, EID, Location, Origin. etc. Fewer or greater, and other data windows can be used in accordance with embodiments of the invention.

FIG. 29 illustrates a window 2900 for managing animal-related data, including selecting a predefined a template. Associated data windows for the window 2900 can include, but are not limited to, templates and description. Fewer or greater, and other data windows can be used in accordance with embodiments of the invention.

FIG. 30 illustrates a window 3000 for managing animal-related data, including creating a batch to process. Associated data windows for the window 3000 can include, but are not limited to, source data, sort by-type criteria, filter by-type criteria, available animals, current herd names, EID, animal status, year code, ear tag number, birth date, origin, breed code, selected animals, EID, working herd, current herd, animal class, animal name, jump date, and comments. Fewer or greater, and other data windows can be used in accordance with embodiments of the invention.

FIG. 31 illustrates a window 3100 for managing animal-related data, including editing a batch to process. Associated data windows for the window 3000 can include, but are not limited to, ranch, herd, batch number, batch revision number, activity date, create date, description, and comments. Fewer or greater, and other data windows can be used in accordance with embodiments of the invention.

FIG. 32 illustrates a window 3200 for managing animal-related data, including selecting from an animal list. Associated data windows for the window 3200 can include, but are not limited to, animals in the herd, EID, last process date, animal status, year code, ear tag number, Brucellosis number, birth date, origin, and primary breed. Fewer or greater, and other data windows can be used in accordance with embodiments of the invention.

FIG. 33 illustrates a window 3300 for managing animal-related data, including searching for activities. Associated data windows for the window 3300 can include, but are not limited to, data filter, data, activity, date from, date to, EID, activity name, activity date, processing, and invalid. Fewer or greater, and other data windows can be used in accordance with embodiments of the invention.

FIG. 34 illustrates a window 3400 for managing animal-related data, including providing a consolidated breeding report. Associated data windows for the window 3400 can include, but are not limited to, company name, ranch name, herd name, beginning date, ending date, report time, breeding year, calf crop year, # exposed, # bred preg check, % bred preg check, # cows marking and branding, # calves marking and branding, % checked marking and branding, % exposed marking and branding, # cows weaning, # calves weaning, % checked weaning, % checked weaning, total production herds, total other herds, and grand total. Fewer or greater, and other data windows can be used in accordance with embodiments of the invention.

FIG. 35 illustrates a window 3500 for managing animal-related data, including providing a herd inventory report. Associated data windows for the window 3500 can include, but are not limited to, company name, ranch name, herd name, description, ending date, report time, total, date established, EID, VID-Ranch, VID-Brucellosis, registration number, classification, breed, purchase date, age in months, date, weight, hip height, and BCS. Fewer or greater, and other data windows can be used in accordance with embodiments of the invention.

While the above description contains many specifics, these specifics should not be construed as limitations on the scope of the invention, but merely as exemplifications of the disclosed embodiments. Those skilled in the art will envision any other possible variations that are well within the scope of the invention.

The invention we claim is:

1. A method for collecting and managing animal-related information, comprising:
receiving previously stored animal-related data;
receiving a selection of at least a portion of the previously stored animal-related data;
generating a data structure adapted to be associated with new animal-related data;
receiving an identifier for an animal, wherein the identifier is associated with the data structure; and
receiving new animal-related data for association with the data structure.

2. The method of claim 1, wherein receiving previously stored animal-related data comprises downloading data from at least one of the following: a server, memory, a database, a data storage device.

3. The method of claim 1, wherein receiving a selection of at least a portion of the previously stored animal-related data comprises user input of a portion of the previously stored animal-related data.

4. The method of claim 1, wherein the data structure comprises at least one of the following: a scan card, an event group.

5. The method of claim 1, wherein the identifier comprises at least one of the following: a RFID tag, a RFID card, a bar code, a radio frequency signal.

6. The method of claim 1, wherein receiving new animal-related data for association with the data structure comprises user input of the new animal-related data.

7. A method for collecting and managing animal-related information, comprising:
receiving animal-related data associated with a plurality of animals;
storing at least some of the received animal-related data;
receiving a request for a portion of the received animal-related data; and
based on at least the request, providing at least some of the received animal-related data.

8. The method of claim 7, wherein receiving animal-related data associated with a plurality of animals comprises receiving information from at least one of the following: a client collection device, a database, a server, a memory.

9. The method of claim 7, wherein storing at least some of the received animal-related data comprises storing the data in at least one of the following: a database, a memory, a data storage device.

10. The method of claim 7, wherein receiving a request for a portion of the received animal-related data comprises receiving at least one of the following: a user selection, a user command, an automatic command, and an automatic selection.

11. The method of claim 7, wherein the animal-related data is associated with at least one event.

12. The method of claim 7, wherein the animal-related data is associated with a plurality of events comprising an event group.

13. The method of claim 7, wherein the animal-related data is associated with at least one unique identifier.

14. The method of claim 7, wherein the animal-related data is associated with at least one animal associated with a RFID device.

15. A method for collecting and managing animal-related information, comprising:
logging in a network user;
receiving a selection of an animal management function;
defining an animal-related group and related data;
defining at least one template based at least in part on animal-related data;
locating at least some of the desired animal-related data;
processing at least some of the desired animal-related data; and
managing at least some of the desired animal-related data.

16. A system for collecting and managing animal-related information, comprising:
a processor adapted to:
receive previously stored animal-related data;
receive a selection of at least a portion of the previously stored animal-related data;
generate a data structure adapted to be associated with new animal-related data;
receive an identifier for an animal, wherein the identifier is associated with the data structure; and
receive new animal-related data for association with the data structure.

17. The system of claim 16, wherein the element adapted to receive previously stored animal-related data comprises to download data from at least one of the following: a server, memory, a database, a data storage device.

18. The system of claim 16, wherein the element to receive a selection of at least a portion of the previously stored animal-related data comprises user input of a portion of the previously stored animal-related data.

19. The system of claim 16, wherein the data structure comprises at least one of the following: a scan card, an event group.

20. The system of claim 16, wherein the identifier comprises at least one of the following: a RFID tag, a RFID card, a bar code, a radio frequency signal.

21. The system of claim 16, wherein the element to receive new animal-related data for association with the data structure comprises user input of the new animal-related data.

22. A system for collecting and managing animal-related information, comprising:
a processor adapted to receive animal-related data associated with a plurality of animals;
a data storage device adapted to store at least some of the received animal-related data;
the processor further adapted to:
receive a request for a portion of the received animal-related data; and
based on at least the request, provide at least some of the received animal-related data.

23. The system of claim 22, wherein the element to receive animal-related data associated with a plurality of animals comprises to receive information from at least one of the following: a client collection device, a database, a server, a memory.

24. The system of claim 22, wherein the element to store at least some of the received animal-related data comprises to store the data in at least one of the following: a database, a memory, a data storage device.

25. The system of claim 22, wherein the element to receive a request for a portion of the received animal-related data comprises to receive at least one of the following: a user selection, a user command, an automatic command, and an automatic selection.

26. The system of claim 22, wherein the animal-related data is associated with at least one event.

27. The system of claim 22, wherein the animal-related data is associated with a plurality of events comprising an event group.

28. The system of claim 22, wherein the animal-related data is associated with at least one unique identifier.

29. The system of claim 22, wherein the animal-related data is associated with at least one animal associated with a RFID device.

30. A method for collecting and managing animal-related information, comprising:
logging in a network user;
receiving a selection of an animal management function;
defining an animal-related group and related data;
defining at least one template based at least in part on animal-related data;
locating at least some of the desired animal-related data;
processing at least some of the desired animal-related data; and
managing at least some of the desired animal-related data.
